(19) United States
(12) Patent Application Publication
(10) Pub. No.: US 2009/0194109 A1
(43) Pub. Date: Aug. 6, 2009

(54) CPAP INTERFACE AND BACKUP DEVICES

(76) Inventors: Rajiv Doshi, Los Altos, CA (US); Arthur Ferdinand, San Jose, CA (US); Elliot Sather, San Francisco, CA (US); Michael L. Favet, San Jose, CA (US); Danny Yu-Youh Lai, San Jose, CA (US); Toru Mino, Chicago, IL (US)

Correspondence Address: SHAY GLENN LLP, 2755 CAMPUS DRIVE, SUITE 210, SAN MATEO, CA, 94403, US

(21) Appl. No.: 12/364,264

(22) Filed: Feb. 2, 2009

Related U.S. Application Data
(60) Provisional application No. 61/025,585, filed on Feb. 1, 2008.

(51) Int. Cl. A61M 16/00

(52) U.S. Cl. 128/204.23; 128/204.18

(57) ABSTRACT

Described herein are combined active PAP/passive EPAP interface devices to transmit positive air pressure from a PAP source to the user, but provide passive EPAP when the PAP source is disabled. These interface device may continue to provide benefit to the user even if the PAP source becomes disconnected or otherwise fails. The interface devices described herein include a passive EPAP airflow resistor configured to provide expiratory positive airway pressure (“EPAP”). These interface devices may also include quick connects and/or disconnects for releasably connecting to the source of pressurized breathable gas, a quick release for disconnecting from the source of pressurized breathable gas, and an adhesive user interface region that connects the device the user's face. Also described are adapter for converting a PAP interface devices into combined active PAP/passive EPAP interface devices, and methods of using these devices.

### CROSS-REFERENCE TO RELATED APPLICATION

This patent application claims priority to U.S. Provisional Patent Application Ser. No. 61/025,585, filed on Feb. 1, 2008. This application is herein incorporated by reference in its entirety.

### BACKGROUND OF THE INVENTION

A Continuous Positive Airway Pressure (CPAP) machine is frequently used for the treatment of sleep apnea and other respiratory and sleep disorders. Apneas, such as obstructive sleep apnea, may occur when the upper airway becomes narrow as the muscles relax during sleep. This reduces oxygen saturation in the blood and causes arousals from sleep. A CPAP machine may help prevent apnea by delivering a stream of air from the CPAP machine through a hose or tube to the user's airway via a CPAP user interface such as nasal pillows, nasal prongs, nasal mask, oral mask or a full-face mask (referred to herein as “CPAP interface devices” or “CPAP only interface devices”). CPAP serves to splint open the airway (keeping it open using positive air pressure) so that unobstructed breathing becomes possible, reducing and/or preventing apneas and hypopneas, and may also help reduce or eliminate snoring.

CPAP treatment can be highly effective in the treatment of obstructive sleep apnea, as long as the user uses the device. Unfortunately, user compliance with CPAP devices is notoriously low. Some users adjust to the treatment within a few weeks, but others struggle for longer periods. Currently available CPAP devices are often uncomfortable, may be noisy and many users have a hard time acclimating to the devices. As a result, users often remove the CPAP mouth/nose interface during a sleep period after wearing the device for only a short period of time, so that they are without CPAP for at least a portion of the night. Furthermore, after CPAP is removed, there is no further therapeutic benefit, and the sufferer resumes having apneas.

Additionally, in many countries where power supply can be unstable or prone to outages, those apnea sufferers who require CPAP are at ask for non-treatment when the electricity is not available to power their CPAP air blowers.

There are numerous types of CPAP devices as well as CPAP interface devices on the market, including masks, headgear, nasal pillows, nasal masks, and the like. Interface devices are described, for example, in U.S. Pat. No. 7,302,950, U.S. Pat. No. 7,287,528, U.S. Pat. No. 7,267,122, and published patent application U.S. 2007/0119454. Such devices have similar compliance problems, however, and may be removed by the user during use, preventing continued therapeutic benefit. Existing interface devices for CPAP sources, such as masks and nasal pillows, generally do not have backup that could continue to provide therapeutic benefit to the user in case the CPAP source device should be shut off, disengaged or otherwise removed during operation.

Thus, it would be beneficial to provide devices and methods that address these problems. In particular, it would be beneficial to provide an interface device having a passive backup (e.g., a passive EPAP function) that may be used in conjunction with device providing active CPAP. Described herein are devices, systems and methods that may address the problems identified above.

### SUMMARY OF THE INVENTION

Described herein are combined active PAP/passive EPAP interface devices for use with systems that generate positive airway pressure (PAP), such as CPAP (or alternatively bi-level PAP, BiPAP, APAP, VPAP or other PAP variants). These devices may provide passive EPAP as a backup therapy in the event that the active PAP source is interrupted or discontinued during application of a therapy. In general, an active PAP/passive EPAP interface device connects a user's airway, such as the mouth and/or nose, to the source of positive airflow, which may be referred to as a PAP source or active PAP source (e.g., a CPAP source). For example, an interface device may be configured as a mask such as a face mask, nasal mask, oral/nasal mask, nasal prong, nasal pillow, or the like. The interface devices described herein include a passive respiratory pressure-regulating airflow resistor that may act as a backup if the active positive pressure applied by a PAP machine is no longer being provided through the interface device. The interface devices described herein are typically worn in conjunction with a PAP supply device, and if the PAP supply is removed during sleep, (e.g., by pulling off the PAP source tubing or by a loss of power), the interface may continue to provide therapeutic benefit by passively creating resistance to expiration while creating only minimal or negligible resistance to inspiration. Although the PAP devices described herein are typically referred to as continuous PAP (or CPAP) devices, these devices may be used with any source of active positive air pressure, including non-continuous positive air pressure devices or other types of ventilators.

The passive expiratory pressure created by the airflow resistor may be referred to as expiratory positive airway pressure (“EPAP”). Thus, the interface devices described herein may be referred to as combined PAP/EPAP interface devices, combined CPAP/EPAP interface devices, active PAP/passive EPAP interface devices, active CPAP/passive EPAP interface devices or the like. For the sake of brevity, in some variations these interface devices are referred to simply as an “interface device,” “interface”, “mask,” “combined CPAP/EPAP interface” or the like. Furthermore, a system including an interface device as described herein along with a source of PAP may be referred to as a combined CPAP/EPAP system, or an active CPAP/passive EPAP system, and the interface devices may be referred to as an “interface device with a passive EPAP backup”, “CPAP backup interface devices,” or as “combined CPAP/EPAP interface devices.”

Also described herein are adapters for PAP interface devices that convert a PAP (e.g., CPAP) only interface device into a combined active PAP/passive EPAP interface device. Thus, a PAP-only interface mask may be converted into a combined PAP/EPAP interface device by the appropriate addition of an adapter. These adapters may include an airflow resistor and an EPAP leak path regulator. The airflow resistor and leak path regulator may increase the resistance to expiration through the interface device when the PAP source is disconnected from the interface, so that the resistance to expiration is in a range that is compatible with passive EPAP (e.g., a therapeutic range), while providing a decreased resistance to inhalation. An adapter may also include an inactivating/activating element for engaging the passive EPAP capability of the mask (typically by engaging or disengaging the passive EPAP airflow resistor) when the source of PAP has shut off or been discontinued. Interface devices including an adapter may also be referred to as merely combined PAP/EPAP interface device, and some of the same features and variations are described below when talking about general active PAP/passive EPAP interface devices.

The combined active PAP/passive EPAP interface devices described herein typically include: (1) a user or user interface body that may include a surface that contacts the user's face or a portion of the user's face (e.g., mouth and/or nose), and may have an air channel that can be secured in communication with a user's airway; (2) a PAP source connector configured to connect a source of pressurized breathable gas to an air channel through the interface body, so that air from the PAP source may be passed through the user interface and to the user; and (3) a passive EPAP airflow resistor configured to inhibit expiration more than inspiration when the source of breathable gas is not supplying pressurized gas to the user, so that the resistance to expiration is within a therapeutic range. The passive EPAP airflow resistor may include or be used in conjunction with an EPAP leak path regulator.

As used herein, the terms “user”, “patient” or “subject” includes any appropriate subject, including human and non-human subjects. A user may be a patient or subject, and may be using the device to receive a therapeutic effect (e.g., treatment of apnea or other sleeping disorder).

In general, the combined active PAP/passive EPAP interface devices described herein include an interface body surrounding an air channel that is to be placed in communication with the user's airway. The interface body may have a sealing surface that seals against the user's face or a portion of the user's face (e.g., around the nose, in the nose, around the mouth and nose, around the jaw, on the cheek, etc.). The interface devices also include a connector to a PAP source. The connector may be an opening that is adjacent to the rest of the interface body, or it may be separated from the interface body by tubing or the like. For example, the connector may include a length of tubing that extends from the interface body. Thus, the connector may be positioned over or around the subject's head. As mentioned above, a combined active PAP/passive EPAP interface device may also include a PAP leak path that may be on any portion of the interface device, including the connector and the interface body. These devices also include a passive EPAP airflow resistor having an EPAP leak pathway. The airflow resistor may include a valve (e.g., flap valve or PEEP valve) that may be either present in the air channel through the interface body (i.e., and disabled or held open during active PAP mode), or may be positioned before the opening(s) into the air channel of the interface device (e.g., between the user and the interface device), or may be positioned near the exit from the air channel of the interface device (e.g., on the connector or between the connector and the interface body). In some variations an EPAP actuator may be included for toggling between the active PAP and passive EPAP modes by activating/inactivating the passive EPAP airflow resistor. In some variations, an EPAP leak pathway regulator may be included, which may at least partially occlude the larger PAP leak pathway during passive EPAP mode. For example, the EPAP actuator may cause a sliding member to cover (or partially cover) openings in the interface body during passive EPAP mode. Thus, the EPAP leak pathway regulator may be a cover or other movable occlusive member. The EPAP leak pathway regulator may be controlled by (or connected to) the EPAP activator.

In general, the therapeutic range provided by the passive EPAP airflow resistor and the EPAP leak pathway is sufficient to provide passive expiratory pressure when the interface device is being worn with the source of PAP disconnected or turned off. For example, the therapeutic range of expiratory resistance(s) provided by the EPAP airflow resistor and EPAP leak pathway may be between 0.001 and about 0.5 cm H₂O/(ml/sec) when the resistance is measured at 100 ml/sec. In some variations, the therapeutic range of expiratory resistance(s) provided by the EPAP airflow resistor and EPAP leak pathway may be between 0.005 and about 0.25 cm H₂O/(ml/sec), or between about 0.01 and about 0.25 cm H₂O/(ml/sec), or between about 0.01 and about 0.20 cm H₂O/(ml/sec), when the resistance is measured at 100 ml/sec. In addition to the range of expiratory airflow resistances, these devices may also keep the resistance to inhalation through the interface device within a predetermined therapeutic range when the active PAP source is turned off or removed. For example, the devices described herein may have a resistance to inhalation when operating in the passive EPAP regime that is within the range of about 0.0001 and about 0.02 cm H₂O/(ml/sec) or between 0.001 and about 0.01 cm H₂O/(ml/sec) when the resistance is measured at 100 ml/sec.

For example, described herein are combined active PAP/passive EPAP interface devices to be worn by a user to transmit positive air pressure from a PAP source to the user that provide passive EPAP backup when the PAP source is disabled. A combined active PAP/passive EPAP interface device may include an interface body that is configured to be secured in communication with the user's airway and to connect to the PAP source to provide PAP (positive airway pressure) to the user, and a passive EPAP airflow resistor in communication with the interface body and configured to passively produce EPAP (expiratory positive airway pressure) when the PAP source is disabled or turned off.

In general, a PAP source may be considered “disabled” when the PAP source no longer provides airflow to the PAP interface. In particular, the PAP source is disabled when it is disconnected, and/or when it is turned off (e.g., loses power). In some variations the PAP source is configured to be intermittent (e.g., to apply PAP during a portion of the respiratory cycle); in such variations, the combined PAP/EPAP devices described herein may be configured to provide passive EPAP only when the PAP source is disabled, and not simply during those portions of the respiratory cycle when positive airway pressure is not being applied. For example, the devices described herein may be configured to apply passive EPAP only when the power (e.g., electrical power) to the PAP source is interrupted or discontinued, or when the PAP source becomes disconnected from the interface.

Thus, in general, the interface devices described herein comprise a connector for connecting to PAP source, a passive EPAP airflow resistor that is configured to create EPAP in the subject when the PAP source is removed or disabled, and an EPAP actuator for enabling the passive EPAP airflow resistor to regulate airflow through the interface when the PAP source is discontinued. As described in greater detail below, the EPAP actuator may detect when the PAP source is disconnected from the interface device, or when the PAP source is not applying positive airflow (e.g., when it is turned off). Thus, the EPAP actuator may be a detector or sensor, e.g., for detecting the presence or absence of positive pressure airflow from the PAP source. The EPAP actuator may connect or receive input from the PAP source directly. For example, the EPAP actuator may be activated when the power to the PAP source is turned off, or when the electrical load on the PAP source indicates that the PAP source has been disconnected. In some variations, the EPAP actuator includes a toggle, switch, displaceable member, or the like, for determining when the combined PAP/EPAP interface device is disconnected from the PAP source. The PAP actuator may include a release mechanism for engaging and/or releasing the passive EPAP airflow resistor to engage or disengage the passive EPAP airflow resistor.

The passive EPAP airflow resistor typically regulates the airflow through or out of the interface device when the PAP source is not functioning or otherwise proving pressurized air to the user. The passive EPAP airflow creates EPAP in a subject wearing the active PAP/passive EPAP interface device. For example, the passive EPAP airflow resistor may include a valve and a leak path that provide resistance to both exhalation and inhalation. When the PAP source is disabled, the interface device functions in the passive EPAP mode. In the passive EPAP mode, the airflow resistor may open during inhalation so that the resistance to inhalation is very low (e.g., between about 0.00001 and 0.02 cm H₂O/(ml/sec), between about 0.00001 and .002 cm H₂O/(ml/sec), etc. when the resistance is measured at 100 ml/sec). The airflow resistor may therefore include a valve such as a valve, a ball valve, or other type of check valve. In the passive EPAP mode during exhalation the airflow resistor may close at least enough so that the resistance to exhalation is higher than the resistance to inhalation and with a range sufficient to create EPAP in the user. For example, during exhalation the airflow resistor may be configured so that the resistance to exhalation is between about 0.001 and about 0.5 cm H₂O/(ml/sec), or between about 0.005 and about 0.25 cm H₂O/(ml/sec), or between about 0.01 and about 0.25 cm H₂O/(ml/sec) when the resistance is measured at 100 ml/sec. The airflow resistor may set the resistance to exhalation by limiting the pathway for air exiting the interface device during exhalation compared to inhalation. The air leaving the interface device during passive EPAP may pass through one or more “leak paths” or leak pathways. Thus, in the sense that the airflow resistor sets the resistance to exhalation and inhalation during EPAP, the airflow resistor may include both a valve and one or more leak paths through the interface device. The leak path is typically a static pathway through the interface device through which air may be exhaled. For example, the leak path may include openings or channel from the air channel through the interface body and/or valve of the airflow resistor that are open during exhalation. More than one leak path may be present, and these leak paths may combine to provide the overall resistance to exhalation during passive EPAP. In some variations, the leak pathway may be adjustable, in order to adjust the resistance to exhalation. Adjustability may be provided by increasing the number and/or size (e.g., cross-sectional area) of the leak path openings.

As mentioned, the interface device may be toggled between passive EPAP and active PAP (e.g., CPAP) by an EPAP actuator. EPAP actuators will be described in greater detail below. During active PAP, positive air pressure is applied to the interface device, and therefore to the user wearing the device. Positive air pressure is typically provided by blowing air through the interface device into the user's airway. Typically, the PAP source may be adjusted to control the applied airflow and/or pressure. During active PAP, the passive airflow resistor that can apply passive EPAP is disabled. The resistance to exhalation through the interface device is generally determined by the flow from the PAP device. During the active PAP mode of operation, the interface device may include one or more leak paths referred to as “PAP leak paths”. PAP leak paths are openings through the interface device through which air may be exhaled during operation in the active PAP mode. These openings may be fixed openings (e.g., holes or perforations) in the interface or tubing connecting to the PAP source. The combined PAP leak path is generally larger (e.g., allowing more exhalation airflow) than the combined passive EPAP leak path. This may be because the exhaled gas includes both the expired gas as well as the gas supplied by the PAP device, in variations such as CPAP.

The PAP leak path during exhalation is typically greater than the EPAP leak during exhalation in the interface devices described herein. During the passive EPAP mode, the resistance to exhalation originates from the passive EPAP airflow resistor (including the leak path openings), while during PAP mode, any resistance to exhalation typically arises largely from the active application of respiratory gas provided by an air blower for example.

Thus, in general, the interface devices described herein may include a PAP leak pathway in communication with the interface body, through which air may be exhaled when the PAP source is enabled and providing positive air pressure though the interface body. An interface device may also include an EPAP leak pathway in communication with the interface body, through which air may be exhaled when the PAP source is disabled, further wherein the EPAP leak pathway permits less airflow through the interface body from exhalation than a PAP leak pathway permits during exhalation when the PAP source is enabled and providing positive air pressure through the interface body.

The combined active PAP/passive EPAP devices described herein may include a connector for connecting the PAP source to the interface. The PAP source (which is typically an air blower) may, in general, be referred to as a source for delivering pressurized breathable gas. In some variations, this connector is a releasable connector, so that the PAP source may be disconnected, or preferentially disconnected, from the interface by the user. In some variations, the interface device includes a quick release mechanism configured to disconnect the PAP source from the interface body. For example, the quick release mechanism may be a pull cord, or the like.

As already mentioned, these devices may also include an EPAP activator configured to inactivate the passive EPAP airflow resistor when the PAP source is enabled, and/or to activate the passive EPAP airflow resistor when the PAP source is disabled. The transition between active PAP and passive EPAP may also include modifying the leak path through the device so that the PAP leak path is partially covered or reduced. For example, one or more openings through the interface device forming the PAP leak path may be covered to form the EPAP leak path portion of the airflow resistor during passive EPAP operation of the interface device.

In some variations, the EPAP activator is configured to inactivate the passive EPAP airflow resistor when the PAP source is connected to the interface body, and/or configured to activate the passive EPAP airflow resistor when the PAP source is disconnected from the interface body. In other variations, the EPAP activator is configured to toggle between the active PAP and passive EPAP modes when the PAP source is powered or unpowered, respectively.

In one example, the EPAP activator comprises a displaceable member configured to be displaced when the PAP source is connected to the interface body. For example, the EPAP activator may be a slideable or bendable member that moves to occlude (e.g., inactivate) all or a portion of the passive EPAP airflow resistor when the PAP source is connected to the interface device. The slideable or bendable member may be a tendon, wire, or the like, that can push against the airflow resistor (including a valve or valve leaflet for example), or can move an airflow resistor out of the way.

In one variation, the EPAP activator includes a displaceable member that moves the airflow resistor in or out of the air channel through the interface device. For example, the EPAP activator may rotate, push, and/or pull an airflow resistor (including a valve such as a flap valve) in or out of the air channel. In other variations the EPAP activator holds the airflow resistor open during active PAP mode, and is withdrawn during passive EPAP mode. For example, if the airflow resistor includes a flap valve configured to close during exhalation through the device, the EPAP activator may hold the flap valve open and disabled during PAP mode.

The combined active PAP/passive EPAP interface devices described herein may be configured as any known type of PAP interface. For example, the interface devices described herein may include a user interface surface configured to contact the user's face that is configured as a nasal pillow, a mask (e.g., nasal mask, an oral/nasal mask, a mouthpiece, etc.) or a nasal prong. In some variations, the interface device may also include one or more attachments to secure the device to the subject's head and/or face. For example, the interface body may include an adhesive surface that is configured to adhesively secure the interface body to the user. The interface device may also include straps or a frame for securing the interface body or any other portion of the device to the user's head. Thus, the interface body may be configured to communicate with both the user's nose and mouth, or with the user's nose but not the user's mouth, etc.

As mentioned above, the passive EPAP airflow resistor may include a flap valve, or other type of valve. For example, the passive EPAP airflow resistor may include a valve selected from the group consisting of: ball valve, flap valve, membrane valve, hingeless valve, balloon valve, duck-bill valve and stopper valve. The passive EPAP airflow resistor may thus provides a resistance to exhalation through the interface body when the PAP source is disabled that is between about 0.001 and about 0.5 cm H₂O/(ml/sec), or between about 0.005 and about 0.25 cm H₂O/(ml/sec), or between about 0.01 and about 0.25 cm H₂O/(ml/sec) when the resistance is measured at 100 ml/sec. The passive EPAP airflow resistor may also include a PEEP or threshold valve. For example, the passive airflow resistor may have a non-zero threshold pressure for opening during expiration so that the airflow resistor is closed during expiration when the pressure across the airflow resistor is below the threshold pressure for opening, and the airflow resistor opens during expiration when the pressure across the airflow resistor exceeds the threshold pressure for opening during expiration.

Also described herein are combined active PAP/passive EPAP interface devices to be worn by a user to transmit positive air pressure from a PAP source to the user (but provide passive EPAP backup when the PAP source is disabled or disengaged) that include: an interface body having an air channel, wherein the interface body is configured to secure the air channel in communication with a user's airway; a connector configured to connect the air channel to a PAP source; and a passive EPAP airflow resistor configured to inhibit exhalation through the air channel more than inhalation through the air channel when the PAP source is not providing pressurized breathable gas through the air channel, wherein the passive EPAP airflow resistor provides a resistance to exhalation through the air channel that is between about 0.001 and about 0.5 cm H₂O/(ml/sec), or between about 0.005 and about 0.25 cm H₂O/(ml/sec), or between about 0.01 and about 0.25 cm H₂O/(ml/sec) when the resistance is measured at 100 ml/sec.

As mentioned, above, the device may include a user interface surface that may be configured as a nasal pillow or a mask, and may be configured to communicate with the user's mouth and/or nose (e.g., or just the user's nose). The active PAP/passive EPAP devices described herein may also include a strap or frame for securing the interface body to the user's head. The connector may be a quick release connector.

As with any of the variations described herein, the passive EPAP airflow resistor may include a flap valve, or may otherwise be selected from the group consisting of: ball valve, flap valve, membrane valve, hingeless valve, balloon valve, duck-bill valve, PEEP, threshold and stopper valve. These interface devices may also include an EPAP leak pathway in communication with the interface body, through which air may be exhaled when the PAP source is disabled. The EPAP leak pathway typically permits less airflow through the interface body during exhalation than the PAP leak pathway permits during exhalation when the PAP source is enabled and providing positive air pressure through the interface body.

Any of the interface devices (and adapter devices for PAP interfaces) described herein may include an EPAP activator configured to inactivate the passive EPAP airflow resistor when the PAP source is enabled and/or to activate the passive EPAP airflow resistor when the PAP source is disabled. The EPAP activator may activate/inactivate the EPAP airflow resistor, and thereby toggle between the active PAP mode and the passive EPAP mode, based on the activity of the PAP source (e.g., power on/power off) or the connection between the PAP source and the interface device (e.g., connected/unconnected to the interface device).

In some variations of the combined active PAP/passive EPAP interface devices described herein, the combined active PAP/passive EPAP interface devices to be worn by a user to transmit positive air pressure from a PAP source to the user (but provide passive EPAP backup when the PAP source is disabled) include: an interface body having an air channel that is configured to be secured in communication with a user's airway; a connector configured to connect to a PAP source in communication with the air channel; a PAP leak pathway through which is exhaled when the PAP source is connected in communication with the air channel; a passive EPAP airflow resistor configured to inhibit exhalation through the air channel more than inhalation through the air channel when the PAP source is disabled; and an EPAP leak pathway through which air is exhaled when the PAP source is disabled, wherein the PAP leak pathway allows greater airflow than EPAP leak pathway. This variation of the active PAP/passive EPAP interface device may include any of the variations as described above.

Also described herein are combined active PAP/passive EPAP interface devices to be worn by a user to transmit positive air pressure from a PAP source to the user (but provide passive EPAP backup when the PAP source is disabled) that include: an interface body having an air channel, wherein the interface body is configured to secure the air channel in communication with a user's airway; a connector configured to connect the air channel to a PAP source; a passive EPAP airflow resistor in communication with the air channel, wherein the passive EPAP airflow resistor is configured to produce expiratory positive airway pressure; and an EPAP activator configured to activate the passive EPAP airflow resistor when the source of PAP is disabled and to inactivate the passive EPAP airflow resistor when the source of PAP is enabled. This variation of the active PAP/passive EPAP interface device may also include any of the variations as described above.

Although the combined PAP/EPAP interface devices described herein typically operate by providing EPAP as a backup once the source of PAP is disabled (e.g., disconnected or otherwise turned off), in some variations the passive resistance provided by the passive EPAP airflow resistor is delayed or ramped up from a low resistance to exhalation to a final (higher) resistance to exhalation. For example, any of the combined PAP/EPAP devices described herein may include an airflow resistor bypass that is configured to transiently decrease the resistance to air exhaled through the passive EPAP airflow resistor for a delay period, after activation of the airflow resistor bypass. An airflow resistor bypass may include a button or other control that can be activated by the user (e.g., by pressing, pulling, etc.) to delay the onset of the passive EPAP for the delay period. In some variations, the airflow resistor bypass may be triggered by the EPAP activator (e.g., upon disabling the PAP source). For example, the airflow resistor bypass may be triggered by disconnecting the PAP source, allowing a delay period before the passive EPAP airflow reaches the fall resistance to exhalation.

An airflow resistor bypass that suspends the operation of the passive EPAP airflow resistor for a delay period may be referred to as a “delay bypass.” In some variations, an airflow resistor bypass includes a bypass channel forming a passageway through which air may pass during exhalation during a delay period, thereby bypassing the airflow resistor. The bypass channel may be the pre-existing PAP leak path, which may be blocked by an EPAP leak path regulator (thus the EPAP leak path regulator may be part of the delay bypass). A bypass channel can be regulated (e.g., opened/closed) by a bypass occluder, so that the bypass channel remains open during the delay period, but is closed (or substantially closed) thereafter. For example, an airflow resistor bypass may include a bypass channel that is located adjacent to the airflow resistor that can be covered by a bypass occluder (e.g., a flap). The bypass occluder acts as a timer. The bypass occluder (or a portion thereof, e.g., a hinge region) will eventually (e.g., after the delay period) return the bypass channel to the closed position, restoring the resistance to exhalation through the device from the airflow resistor. In some variations the bypass occluder is made (at least in part) of a material having a slow recovery from elastic deformation. Thus, the material can be displaced from an original shape configured to obstruct the bypass channel, and gradually return to the original shape to close the bypass channel.

In some variations, the airflow resistor bypass disengages the passive EPAP airflow resistor and prevents or reduces the resistance to exhalation for at least the delay period. Thus, an airflow resistor bypass may include a bypass displacer for displacing all or a portion of the airflow resistor during the delay period. For example, the airflow resistor bypass may include a bypass displacer configured as a bypass hinge that is connected to at least a portion of the airflow resistor. The bypass displacer can be activated to move the airflow resistor at least partially away from the passageway, permitting exhalation through the passageway that is unregulated by the airflow resistor. The airflow resistor bypass may move a valve portion (e.g., flap or flaps) of the airflow resistor out of the passageway. In some variations, the airflow resistor bypass acts by holding the valve of the airflow resistor open (or partially open) for the delay period. For example, the airflow resistor bypass may include a bypass displacer that holds the airflow resistor in an open configuration. In some variations, the airflow resistor bypass disables the airflow resistor in other ways. For example, the airflow resistor bypass may be configured to include a bypass displacer that prevents the valve limiter of an airflow resistor from holding the airflow resistor closed during exhalation. A bypass displacer may move a flap valve support(s) so that it does not engaged the flap valve in the closed position during exhalation. After the delay period, the bypass displacer disengages and the airflow resistor again provides an increased resistance to airflow during exhalation.

The bypass displacer may be an adhesive or other material that releasably secures all or a portion of the airflow resistor and releases it after the delay period. For example, a bypass displacer may be an adhesive that holds a flap of a flap-valve type airflow resistor in an open position until the adhesive releases the flap. The adhesive may be selected so that it releases after an appropriate delay. In some variations, an airflow resistor bypass may also be configured to expand the opening through which air may pass through the nasal device. For example, a nasal device may include a leak pathway that is typically open even during exhalation; an airflow resistor bypass may temporarily enlarge or increase the leak pathway.

The delay period of the delay bypass may be greater than 5 minutes, greater than 10 minutes, greater than 15 minutes, etc. The delay period may be less than 4 hours, less than 3 hours, less than 2 hours, less than 1 hour, or the like. Any appropriate delay period may be used, and the delay period may be fixed, or variable. In general, the delay period may be sufficiently long to allow a subject to fall asleep with the device, so that the airflow resistor becomes active after the patient falls asleep.

Other examples of delay bypasses (airflow resistor bypasses) may be found in co-pending U.S. patent application Ser. No. 12/329,895, which is herein incorporated by reference in its entirety.

Adapters for converting PAP interface devices into combined active PAP/passive EPAP interface devices are also described. In general, and adapter may be an adapter system, including a plurality of components that may be connected to a PAP interface device, or an adapter may be a single component adapter that can be connected to the PAP interface device. Various examples of adapters are provided and described below, and may include attachment to the subject as well as the PAP interface device.

For example, described herein are adapter device for a PAP interface that connects to a PAP source. The adapter device is capable of converting the PAP interface into a combined active PAP/passive EPAP interface that provides passive EPAP backup when the PAP source is disabled. The adapter device may include: an EPAP passive airflow resistor configured to be placed in communication with an airway through the PAP interface to passively inhibit exhalation more than inhalation through the PAP interface and produce expiratory positive airway pressure; and an EPAP actuator configured to activate the passive EPAP airflow resistor when the PAP source is disabled and to inactivate the passive EPAP airflow resistor when the PAP source is enabled.

In some variations, the adapter device (or system) includes components having the EPAP airflow resistor and/or the EPAP actuator that are swapped for components of an existing PAP adapter device. For example, a CPAP nasal mask (or nasal pillow, nasal prong, face mask, etc.) may include a region of tubing between the adapter body (e.g., having an air channel therethrough) and a connector configured to connect to the PAP source. An adapter device may convert the PAP interface to a combined PAP/EPAP interface by swapping out a region of the tubing, or by attaching a new region of tubing that will be located between the interface device and the source of PAP. This adapter body region (in this example, a region of tubing), may include an air passage that is configured to be placed in communication with the airway through the PAP interface, and the EPAP airflow resistor may be located therein. The same (or a separate) region of tubing may include an EPAP actuator for enabling/disabling the EPAP airflow resistor. The EPAP activator comprises a sensor to determine when the flow of positive pressure from the PAP source has been discontinued. The EPAP activator may comprise a displaceable member configured to be displaced when the PAP source is connected to the PAP interface.

In some variations, the adapter device includes a disconnect region that provides a preferential region for disconnecting the PAP source from the rest of the interface.

In some variations, the adapter device also includes an EPAP leak path regulator configured to reduce the exhalation leak pathway through the device when the PAP source is disabled. For example, the leak path regulator may be a sliding cover that covers some of the PAP leak paths (e.g., holes through a portion of the PAP interface) after the PAP source is disabled.

The passive EPAP airflow resistor may be configured to provide a resistance to exhalation through the PAP interface when the PAP source is disabled that is in any of the therapeutic ranges for inhalation and/or exhalation described above. For example, the adapter may be configured to provide a passive EPAP airflow resistor having a resistance to exhalation between about 0.001 and about 0.5 cm H₂O/(ml/sec), or between about 0.005 and about 0.25 cm H₂O/(ml/sec), or between about 0.01 and about 0.25 cm H₂O/(ml/sec), when the resistance is measured at 100 ml/sec.

Another variation of an adapter device (or system) for a PAP interface that connects to a PAP source (capable of converting the interface into a combined active PAP/passive EPAP interface that provides passive EPAP backup when the PAP source is disabled) includes: an EPAP passive airflow resistor configured to be placed in communication with an airway through the PAP interface to passively inhibit exhalation more than inhalation through the PAP interface and produce expiratory positive airway pressure when the PAP source is disabled; an EPAP leak path regulator configured to reduce the exhalation leak pathway through the device when the PAP source is disabled; and an EPAP actuator configured to activate the passive EPAP airflow resistor and the EPAP leak path regulator when the PAP source is disabled and to inactivate the passive EPAP airflow resistor and the EPAP leak path regulator when the PAP source is enabled.

As mentioned above, any of the adapter devices described herein may also include an adapter body having an air passage that is configured to be placed in communication with the airway through the PAP interface. The adapter body may be configured as a tubing component, or a ‘swappable’ region of a PAP interface device that may be connected to, or may swap out, a portion of the PAP interface device. Thus, the adapter device (or system) may include one or more adhesive connectors, friction fits, threaded (screw) fits, or the like, for connecting to the PAP interface device that is being adapted to a combined PAP/EPAP device.

Also described herein are methods of treating sleeping disorders using combined PAP/EPAP devices. For example, a method of treating a sleeping disorder may include the steps of: providing an active PAP therapy by placing a PAP source in communication with a user's airway through an active PAP/passive EPAP interface connected to the PAP source; and passively creating EPAP in the subject using the active PAP/passive EPAP interface when the PAP source is disabled. The method may also include the step of securing the active PAP/passive EPAP interface in communication with the user's mouth and nose, or of securing the active PAP/passive EPAP interface in communication with the user's nose.

In some variations, the step of passively creating EPAP in the subject using the active PAP/passive EPAP interface when the PAP source is disabled comprises passively creating EPAP in the subject using the active PAP/passive EPAP interface when the PAP source is removed from the active PAP/passive EPAP interface.

The step of passively creating EPAP in the subject may comprise activating a passive EPAP airflow resistor when the PAP source is disabled.

In some variations, the step of passively creating EPAP in the subject comprises providing a resistance to exhalation through the PAP interface when the PAP source is disabled that is within any of the therapeutic ranges described above (e.g., between about 0.001 and about 0.5 cm H₂O/(ml/sec), or between about 0.005 and about 0.25 cm H₂O/(ml/sec), or between about 0.01 and about 0.25 cm H₂O/(ml/sec), when the resistance is measured at 100 ml/sec). In some variations, the method may include the step of providing a resistance to inhalation that is within any of the therapeutic ranges describe above. In some variations, the resistance to inhalation is negligible or minimal.

Another method of treating a sleeping disorder includes the steps of: placing an active PAP/passive EPAP interface device in communication with a subject's airway; applying a positive air pressure through the interface device by placing the interface device in communication with a PAP source; and passively creating EPAP in the subject by inhibiting exhalation more than inhalation through the interface device when the positive air pressure from the PAP source is disabled.

The step of placing an active PAP/passive EPAP interface device in communication with a subject's airway may include securing the active PAP/passive EPAP interface in communication with the user's mouth and nose, or securing the active PAP/passive EPAP interface in communication with the user's nose.

In some variations, the step of passively creating EPAP in the subject by inhibiting exhalation more than inhalation through the interface device when the positive air pressure from the PAP source is disabled may include passively creating EPAP in the subject using the active PAP/passive EPAP interface when the PAP source is removed from the active PAP/passive EPAP interface. Alternatively, the step of passively creating EPAP in the subject may comprise activating a passive EPAP airflow resistor when the PAP source is disabled (including turned off).

As mentioned above, the step of passively creating EPAP in the subject comprises providing a resistance to exhalation through the PAP interface when the PAP source is disabled that is within any of the therapeutic ranges described above (e.g., between about 0.001 and about 0.5 cm H₂O/(ml/sec), or between about 0.005 and about 0.25 cm H₂O/(ml/sec), or between about 0.01 and about 0.25 cm H₂O/(ml/sec), when the resistance is measured at 100 ml/sec). In some variations, the method may include the step of providing a resistance to inhalation that is within any of the therapeutic ranges describe above.

Also described herein are methods of converting a PAP interface device into a combined active PAP/passive EPAP interface device. For example, the method may include the steps of: providing a PAP interface device configured to connect to a PAP source; and attaching an EPAP passive airflow resistor in communication with an airway through the PAP interface device so that the EPAP passive airflow resistor passively inhibits exhalation more than inhalation through the PAP interface to create EPAP in a user when a PAP source is not applying positive air pressure through the interface to the user.

The method may also include the step of attaching an EPAP actuator to the PAP interface device, wherein the EPAP actuator is configured to activate the passive EPAP airflow resistor when the PAP source is disabled and to inactivate the passive EPAP airflow resistor when the PAP source is enabled. In some variations, the method includes the step of attaching a leak path regulator to the PAP interface device, wherein the leak path regulator is configured to reduce the exhalation leak pathway through the device when the PAP source is disabled.

The step of attaching an EPAP passive airflow resistor in communication with an airway through the PAP interface device comprises securing an EPAP passive airflow resistor that its configured to provide a resistance to exhalation through the PAP interface device when the PAP source is disabled that is within any of the therapeutic ranges described herein, such as between about 0.001 and about 0.5 cm H₂O/(ml/sec), or between about 0.005 and about 0.25 cm H₂O/(ml/sec), or between about 0.01 and about 0.25 cm H₂O/(ml/sec), when the resistance is measured at 100 ml/sec.

Also described herein are methods of converting a PAP interface device into a combined active PAP/passive EPAP interface device including the steps of: attaching an EPAP passive airflow resistor in communication with an airway through the PAP interface device so that the EPAP passive airflow resistor passively inhibits exhalation more than inhalation through the PAP interface device when a PAP source is not applying positive air pressure through the PAP interface device; and attaching an EPAP actuator to the PAP interface device, wherein the EPAP actuator is configured to activate the passive EPAP airflow resistor when the PAP source is disabled and to inactivate the passive EPAP airflow resistor when the PAP source is enabled.

As mentioned above, the method of converting a PAP interface into a combined PAP/EPAP interface may also include attaching a leak path regulator to the PAP interface device, wherein the leak path regulator is configured to reduce the exhalation leak pathway through the PAP interface device when the PAP source is disabled.

The step of attaching the EPAP passive airflow resistor in communication with an airway through the PAP interface device comprises securing an EPAP passive airflow resistor that its configured to provide a resistance to exhalation through the PAP interface device when the PAP source is disabled that is within any of the therapeutic ranges described herein, such as between about 0.001 and about 0.5 cm H₂O/(ml/sec), or between about 0.005 and about 0.25 cm H₂O/(ml/sec), or between about 0.01 and about 0.25 cm H₂O/(ml/sec) when the resistance is measured at 100 ml/sec. In some variations, the method may also include providing a resistance to inhalation that is within the therapeutic range for a resistance to inhalation during passive EPAP, as described herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### BRIEF DESCRIPTION OF THE DRAWINGS

FIGS. 1A-1C are schematic illustrations of combined PAP/EPAP interface devices.

FIG. 2A is a perspective view of a combined active PAP/passive EPAP interface device.

FIG. 2B is a front perspective view of the combined active PAP/passive EPAP interface device of FIG. 2A shown being worn on a user's head.

FIG. 2C is a side perspective view of the combined active PAP/passive EPAP interface device of FIG. 2A shown being worn on a user's head.

FIG. 2D is a cross-sectional view of the combined active PAP/passive EPAP interface device of FIG. 2A shown connected to a PAP source.

FIG. 2E is a cross-sectional view of the combined active PAP/passive EPAP interface device of FIG. 2A shown disconnected from a PAP source during inhalation.

FIG. 2F is a cross-sectional view of the combined active PAP/passive EPAP interface device of FIG. 2A shown disconnected to a PAP source during exhalation.

FIG. 3A is a perspective view of a combined active PAP/passive EPAP interface device.

FIG. 3B is a front view of a portion of the combined active PAP/passive EPAP interface device shown in FIG. 3A.

FIGS. 3C-3E are cross-sections through the region of the combined active PAP/passive EPAP interface device shown in FIG. 3A connected to a PAP source, disconnected from the PAP source during inhalation, and disconnected from the PAP source during exhalation, respectively.

FIG. 4A is a perspective front view of a combined active PAP/passive EPAP interface device connected to a PAP source.

FIG. 4B shows the combined active PAP/passive EPAP interface device of FIG. 4A disconnected from the PAP source.

FIG. 5A is a perspective front view of another variation of a combined active PAP/passive EPAP interface device connected to a PAP source.

FIG. 5B shows the combined active PAP/passive EPAP interface device of FIG. 5A disconnected from the PAP source.

FIG. 5C is an enlarged view of a portion of a combined active PAP/passive EPAP interface device.

FIG. 6 is a perspective view of one variation of a combined active PAP/passive EPAP interface device.

FIG. 7 is a perspective view of another variation of a combined active PAP/passive EPAP interface device.

FIG. 8 illustrates a combined PAP/EPAP interface device, as described herein.

FIG. 9 is a combined PAP/EPAP interface device similar to the device shown in FIG. 8.

FIG. 10 is another schematic illustration of a combined PAP/EPAP interface device.

FIG. 11 is another variation of a combined PAP/EPAP interface device.

FIG. 12 is another variation of a combined PAP/EPAP interface device.

FIG. 13 illustrates one variation of a combined PAP/EPAP interface device having a quick release mechanism.

FIG. 14 shows an exploded view of a PAP system including an adapter device capable of converting the PAP interface into a combined active PAP/passive EPAP interface that provides passive EPAP backup when the PAP source is disabled.

FIG. 15 illustrates a PAP interface system including an adapter device so that it can function as a combined PAP/EPAP system.

FIGS. 16 and 17 are quick release connectors that may be used as part of the interface devices described herein.

FIG. 18 is a threaded connector.

FIG. 19 is a combined PAP/EPAP interface device in which the passive EPAP airflow resistor is a PEEP valve.

### DETAILED DESCRIPTION OF THE INVENTION

The devices described herein are interfaces for connecting a user's airway to a source of pressurized breathable gas (e.g., a CPAP source). Thus, these interface devices may connect the user's nose or nasal passages or the user's nose and also the user's mouth, or the user's mouth, with an active PAP source that provides pressurized breathable gas. The interface devices described herein also include a passive mechanism that acts as a backup in the event that the active CPAP source is interrupted or otherwise discontinued (i.e. if the user knowingly or involuntarily attempts to remove the active positive airway pressure treatment during sleep). The passive mechanism is an airflow resistor configured to provide expiratory positive airway pressure (“EPAP”). Thus, these devices may be referred to as combined active PAP/passive EPAP interface devices, or PAP/EPAP interface devices. These PAP/EPAP interface devices may also include venting (e.g., PAP leak path(s) and/or EPAP leak path(s)), quick connects and/or disconnects for releasably connecting to the source of pressurized breathable gas, a quick release for disconnecting from the source of pressurized breathable gas, and a user interface region (e.g., an adhesive interface region or other types of non-adhesive interface) that connects the device to the user's face.

In general the combined PAP/EPAP interface devices described herein include: an interface body having a user interface surface that may be configured to secure and/or seal against the user, and an air channel that is configured to communicate with the user's airway and through which breathable gas is passed. These devices may also include a connector configured to releasably connect to a source of pressurized breathable gas (e.g., a PAP source such as a CPAP source). The combined PAP/EPAP devices describe herein also include a passive EPAP airflow resistor that is configured to inhibit expiration more than inspiration when the source of pressurized breathable gas is not supplying pressurized gas to the user through the air channel. These devices may also include an EPAP actuator to activate the passive EPAP mode when the PAP supply is discontinued, for example, by removing the connection to the PAP source. In some variations, the combined PAP/EPAP devices also include an EPAP leak path regulator that reduces the leak path when the PAP supply is discontinued, so that the airflow resistor can apply a therapeutic range of pressure during exhalation to create EPAP in the user.

The interface devices described herein may be used as a user interface with any appropriate positive-pressure supplying device, including but not limited to commercially available PAP devices including (but not limited to): CPAP, APAP, AutoPAP or AutoCPAP, VPAP™, BiPAP®, and xPAP ST devices. Any ventilator that produces airflow using a blower may be included for use with the devices and methods described herein.

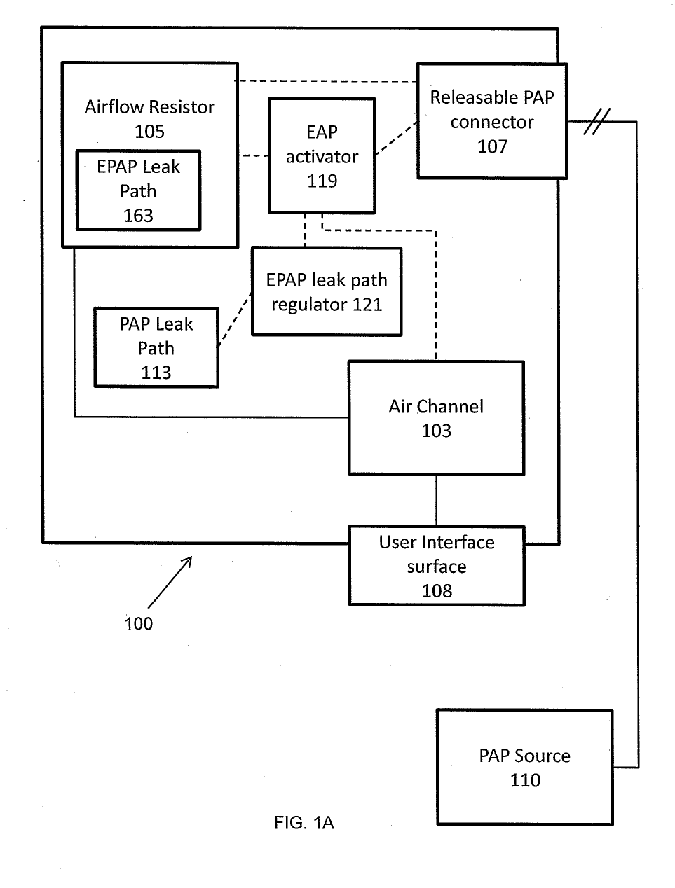

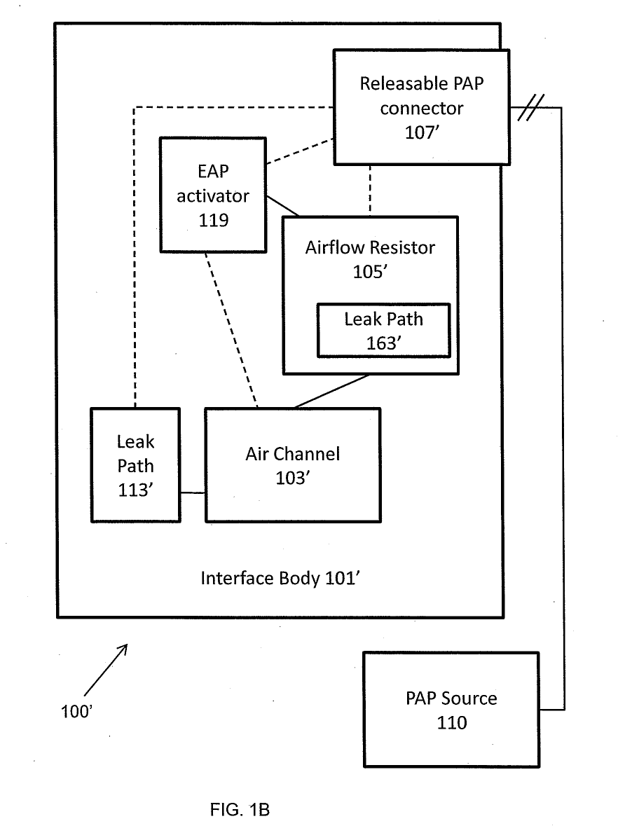

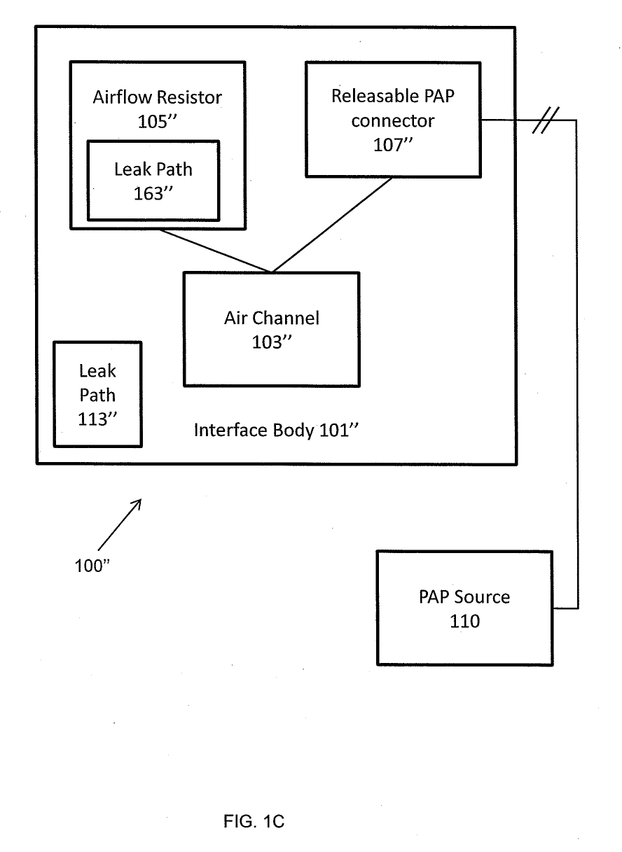

FIGS. 1A-1C schematically illustrate generic combined active PAP/passive EPAP interface devices 100. These interface devices 100 are configured to connect the user's airway to the source of positive air pressure (e.g., PAP source 110). This connection may be through a user interface surface on the interface body. The user interface surface may be adapted for contact with the users. For example, it may be a non-irritating surface (e.g., made or coated with a hypoallergenic material), it may be made from a soft, pliable or compressible material, or the like. The user contacting surface may be configured to seal against the user. The user contacting surface may limit or prevent uncontrolled airflow (e.g., leaks). In some variations, the user contacting surface includes an adhesive that helps secure the device against the user and may help maintain a seal.

An interface device 100 may include an interface body that is configured as a mask, face mask, nasal mask, oral-nasal mask, nasal cushion, nasal pillows or prongs, mouthpieces or the like. Thus, the interface device (particularly the user interface surface) contacts the user's face (or a portion of the face) when the device is worn. The device may be worn at least partly in, over or around the user's nostrils, and may also cover (or partially cover) the user's mouth. In addition to the adhesive attachment means just described, any other appropriate securing means may be used to secure the device to the user. For example, straps, headgear, elastic tethers, belts, or any other anchoring or attachment mechanism may be used to secure the interface device in communication with a user's airway(s).

The interface (e.g., the interface body, the connector, the airflow resistor) may be formed of any appropriate material (e.g., plastics, metals, etc.) or combination of materials. For example, materials forming the device (or forming a layer or portion of the device) may be synthetic and biocompatible polymers (such as thermoplastic elastomers, silicone elastomers, styrene block copolymers, thermoplastic copolyesters, thermoplastic polyamides, thermoplastic polyolefins, thermoplastic polyurethanes, thermoplastic vulcanizates, polyvinyl chloride, fluoropolymers, PTFE, modified PTFE, FEP, ETFE, PFA, MFA, polyurethane, polycarbonates, silicones, acrylic compounds, thermoplastic polyesters, polypropylene, low density polyethylenes, nylons, sulfone resins, high density polyethylenes, etc.), natural polymers (cellulose polymers, collagen, starch blends, hyaluronic acid, alginates, carrageenan, etc.), metals including biocompatible metals (e.g., precious metals including gold and silver, stainless steel, titanium, etc), ceramics (e.g., porcelain, alumina, hydroxyapatite, zirconia), and the like.

The interface 100 typically includes an air channel 103 (or passageway) that is placed in communication with the user's air passages (e.g., one or more of the user's nostrils, mouth, ostomy), when the device is worn. This air channel 103 may be enclosed (e.g., tubular), or it may be open (e.g., a cavity), and is held against the subject when the device is worn. Although many of the embodiments describe herein refer to the air channel as a “passageway”, it should be understood that this is not limited to tubular or otherwise enclosed air channels. The air channels described herein may form an enclosed space when worn by the user, but may be open (or semi-open) when not worn.

An airflow resistor 105 is attached to the interface so that the airflow resistor 105 is in communication with the air channel 103. The airflow resistor is typically configured so that the airflow resistor 105 has a greater resistance to expiration than to inspiration when the device is worn by the subject in the absence of application of PAP from an external source. In general, the airflow resistor is a passive resistance element, meaning that the resistance to expiration does not arise because of the application of airflow (e.g., from pressurized gas). Examples of airflow resistors configured to have a greater resistance to expiration than to inspiration may be found in any the following U.S. patent applications, each of which is incorporated herein in its entirety: U.S. patent application Ser. No. 11/298,640 (titled “NASAL RESPIRATORY DEVICES”) filed Dec. 8, 2005; U.S. patent application Ser. No. 11/298,339 (titled “RESPIRATORY DEVICES”) filed Dec. 8, 2005; U.S. patent application Ser. No. 11/298,362 (titled “METHODS OF TREATING RESPIRATORY DISORDERS”) filed Dec. 8, 2005; U.S. patent application Ser. No. 11/805,496 (titled “NASAL RESPIRATORY DEVICES”) filed May 22, 2007; U.S. patent application Ser. No. 11/811,339 (titled “NASAL DEVICES”) filed Jun. 7, 2007; U.S. patent application Ser. No. 11/759,916 (titled “LAYERED NASAL DEVICES”) filed Jun. 7, 2007; and U.S. patent application Ser. No. 11/811,401 (titled “NASAL RESPIRATORY DEVICES FOR POSITIVE END-EXPIRATORY PRESSURE”) filed Jun. 7, 2007.

For example, an airflow resistor 105 may include a flap valve. A flap valve typically includes one or more valve leaflets. A flap valve may be made of a stiff or flexible layer (e.g., silicone or polyurethane, or any other appropriate material, including flexible materials) that forms one or more movable flaps. A flap valve layer may include a plurality of valve leaflets. The flap valve may also include a valve limiter that limits the motion of the flap(s) so that they are open during inhalation and closed during expiration (or substantially closed). The limiting layer may prevent the flap valve from opening in one direction (e.g., exhalation) by supporting one side of the flap valve. Thus, a limiter may be a mesh, a bar, a post, or the like. One particular type of flap valve includes a plurality of valve leaflets that open from a central point, and the flap valve limiter is a mesh or support that is present on one side of the flap valve. For example, the flap valve may be formed by radial cuts (or spokes) that radiate from a single point (or region) to form triangular leaflets.

Other types of airflow resistors include ball valves, membrane valves, hingeless valves, balloon valves, duck-bill, PEEP, threshold and stopper valves. Each of these examples of airflow resistors at least partially inhibits the passage of airflow during expiration when oriented in communication with the user's nasal passages (and possibly mouth).

More than one airflow resistor 105 may be used. For example, in some variations, the airway interface is connected to two airflow resistors (e.g., cone in communication with each of the user's nostrils).

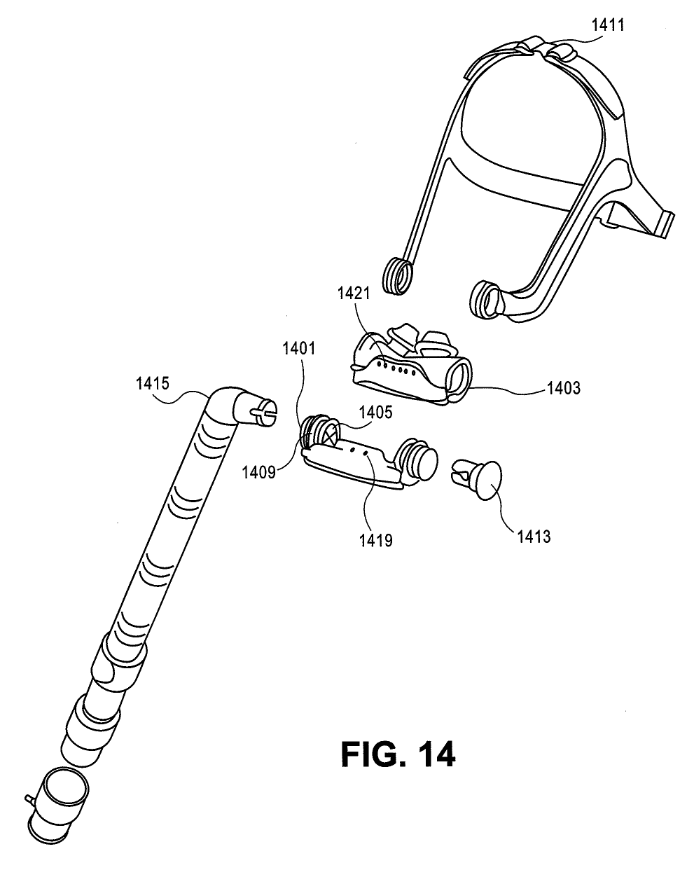

The airflow resistor 105 may be disabled when the source of pressurized gas 110 is attached, and then activated when the source of pressurized gas is detached. In some variations, the airflow resistor is disabled when pressurized gas is being provided and activated when no pressurized gas is provided. For example, the connector and/or the source of pressurized gas 110 may directly interface with the airflow resistor 105 to disengage or inactivate the airflow resistor (e.g., so that it is held “open” during expiration). The airflow resistor may be disabled by propping it open (e.g., using an EPAP activator including a prong, projection, probe, or the like) or by preventing it from closing during expiration (e.g., using an adhesive, grasper, or other means to secure the airflow resistor in the open state). An EPAP activator may also be referred to as an “EPAP inactivator.” In some variations the airflow resistor is always active, regardless of whether or not pressurized gas is being provided to the interface device 100, and no EPAP actuator is required. An example of this is shown below in FIG. 14.

An interface device 100 may also include a connector 107 that is configured to connect to a source of pressurized gas (e.g., a CPAP source) 110. The connector links the PAP source 110 with the interface device, and thus to the user's airway through the air channel 103 through the interface device. The connector 107 may be connected directly to the air channel 103, as discussed below for FIG. 1C, or it may be connected through an airflow resistor 105, as discussed below for FIG. 1B. The passive EPAP airflow resistor (or resistors) 105 may also be coupled or linked to the connector 107 even when the connector is connected to the air channel 103 directly. The connection between the passive EPAP airflow resistor and the connector to the PAP source may be an EPAP activator 119. For example, the EPAP activator may be the coupling between the airflow resistor and the releasable PAP connector, so that a portion of the PAP source contacts the airflow resistor and holds it open when the PAP connector is engaged with the PAP source. In some variations the EPAP activator includes a movable member that is displaced by the connector or the PAP source, when the connector is engaged by the PAP source; displacing the movable member holds the EPAP airflow resistor(s) open or inactive. Removing the connection between the EPAP airflow resistor and the PAP source releases the movable member of the EPAP activator, allowing the passive EPAP airflow resistor to function.

In some variations, the EPAP activator detects the power state of the PAP source, and/or the positive pressure being applied by the PAP source. For example, the EPAP activator may be electrically connected to the PAP source, so that if the power to the PAP source is interrupted, the EPAP activator can activate the passive EPAP airflow resistor. In some variations, the EPAP activator detects the application of positive air pressure from the PAP source. For example, the EPAP activator may communicate with the air channel through the device, and activate the EPAP airflow resistor when the PAP device stops supplying positive air pressure.

As indicated in FIG. 1A, the EPAP activator 119 may receive input from the connector 107, or from the air channel 103, as described above. In some variations, a separate EPAP activator is not required, and the connector 107 directly engages the airflow resistor 105 to activate/inactivate it depending on the presence of the PAP source (e.g., tubing). The EPAP activator may communicate with an EPAP leak pathway regulator 121 that can modify the PAP leak path. For example, if the interface device 100 includes passages or openings (leak paths) that are available for exhalation during the application of PAP, when the PAP source is disabled, these openings (particularly if they are between the airflow resistor and the user interface 101) may be modified or eliminated so that the leak path is determined by the airflow resistor 105 leak path (the EPAP leak path 163). The PAP leak path may be reduced (e.g., by partially or completely occluding the leak path openings) so that the leak path when the PAP source is inactive is the EPAP leak path 163.

Thus, in general, the airflow resistor 105 may be inactivated (e.g., held open) when a source of pressurized gas 110 is attached to the connector, and activated (to resist expiration more than inspiration) when the source of pressurized gas 110 is detached.

The connector 107 may be a releasable connector. For example, the connector may be a quick release and/or quick connect connector. A quick release connector may also be referred to as an “easy release” connector that is configured to break the connection between the CPAP source and the interface device. For example, a connector may connect the air channel to the source of pressurized breathable gas 110 by a friction fitting or other connection that can be readily disengaged by a user, particularly while the user is sleeping. Examples of quick releases include friction-fit connectors, adhesive connectors, Velcro connectors, snap-on connectors, magnetic connectors, press-fit connectors, and clamping connectors. Screw-on connectors may also be configured as quick release connectors.

For example, a connector may be configured to mate with a source of pressurized breathable gas 110 that includes a tube or hose that extends from the source. When the connector is configured as a friction fitting, the tube from the source may slide into (or over) a connector dock. Friction between the dock on the connector and the tube from the source helps hold the source in position. In some variations, additional material (Velcro, snaps, stays, magnets, etc. help to keep the tube attached to the connector. Although the tube may resist being accidentally dislodged from the interface device during use, a quick-release connector may be configured so that only a small amount of additional force is necessary to disengage the source.

In some variations, Velcro or some other releasable material helps secure the tube from the source of breathable gas to the connector. For example, the connector may include an adhesive surface that contacts a mating surface on the tube (or vice-versa). In some variations, a quick-release connector includes a frangible or releasable component that may be broken or released by the user to release the tube from the interface after it has been attached. For example, the connector may include clasps or stays that can be broken or unfastened by applying sufficient force.

A quick-release connector may also include a button, pull, tab, handle, or any other control to trigger the release of or disconnect the source of pressurized gas (CPAP source 110) from the interface device 100. For example, pulling a “rip cord” may quickly release the CPAP source 110 from the connector 107, allowing the airflow resistor 105 to passively resist expiration. A quick release connector may also be a quick attach connector.

The interface devices 100 described herein may also include a user interface surface 108 that is configured to contact the user when the device is worn. For example, the user interface surface may be configured as a nasal mask that fits over and/or around the user's nose, or a nasal prong, that fits into the user's nose, or a face mask that fits over and/or around both the mouth and nose.

As mentioned, any appropriate PAP source 110 may be used with the devices described herein.

FIGS. 1B and 1C are schematic illustrations of other interface devices 100′, 100″, similar to the schematic shown in FIG. 1A. Any of the elements shown in FIG. 1A may be included in FIG. 1B and 1C or may be missing, and arranged as appropriate and described herein. For the sake of simplicity, certain elements (e.g., the user surface interface, the EPAP activator and the EPAP leak path regulator) are not shown. In FIG. 1B, the source of pressurized breathable gas (CPAP source 110) creates airflow that enters the user's airway through the airflow resistor 105′ before entering the air channel 103′. In this example, the connector 107′ is connected to an interface body 101′, and communicates with the airflow resistor 105′. For example, the connector 107′ may engage the airflow resistor 105′ and inactivate it. A tube or hose from the source of pressurized air 110 may connect to the connector 107′ so that air flows from the source of pressurized air 110 and through the airflow resistor on the way to the user's airway. As mentioned above, this may disable the airflow resistor 105′ (so that the airflow resistor cannot increase the resistance to expiration). The connector 107′ may be directly coupled to the airflow resistor 105′ so that the hose or tube from the source 110 may directly disable the airflow resistor.

The interface body 101′ may be any appropriate body to which the airflow resistor and/or connector can attach. The interface body typically forms the air channel. As in the previous example, the interface body may include a user interface surface (not shown) and the user interface surface and interface body region may be configured as a mask, nasal pillow, etc.

In some variations, when the source 110 engages the connector 107′, it disables the airflow resistor through an EPAP activator. The EPAP activator may inactivate the airflow resistor by propping it open, or moving it out of the exit pathway for air to exit the interface device. The EPAP activator may be configured include a sliding rod, piston, tube, (or any other appropriate element) that can project into the airflow resistor to hold it in the open configuration. For example, a spring-biased rod may be displaced when the source 110 engages the connector 107′ so that it disables the airflow resistor. When the source is removed, the spring bias returns the rod to a non-disrupting position in which the airflow resistor is allowed to operate as described. A similar mechanism may be used to disable the airflow resistor even when the connector 107′ is not directly connected to the airflow resistor 105′, as shown in FIG. 1 C.

FIG. 1C illustrates another variation of an interface device 100″ in which the airflow resistor 105″ and the connector 107″ are both in communication with the passageway 103″ of an interface body region 101″, but not necessarily in contact with each other. For example, one or more airflow resistors 105″ may be in fluid communication with the air channel through which pressurized air from the source 110 is supplied to a user's airway. The airflow resistor may be activated or inactivated by the absence or presence of pressurized air from the source 110, as mentioned above, or the airflow resistor may be continuously active, even when the source is supplying pressurized gas.

In the example shown schematically in FIG. 1C, the airflow resistor opens to the external environment (e.g., outside of the interface device) through an opening that is separate from the connector opening 107″ from which air is supplied when the source 110 is connected. When the source 110 is disconnected, the connector may remain “open”—allowing air to enter the air channel 103″ of the interface body 101″, or it may be automatically closed off by a valve (e.g., a gate valve, etc.). Closing off the opening of the connector 107″ may help the airflow resistor 105″ regulate the airflow into the passageway 103″ of the airway interface 101″. In some variations, it is not necessary to close the opening of the connector 107″ when the source 110 is disconnected, since the airflow resistor will still passively increase resistance to exhalation (particularly when the opening through the connector 107″ is relatively small, though of sufficient size for sufficient air supplied by the pressurized source 110.

This is in contrast to the example described in FIG. 1B, in which the opening of the connector 107′ communicates with the air channel 103′ through the valved (or metered) opening of the airflow resistor 105′. In this example, it is not necessary to add an additional valve on opening of the connector 107′.

Any of the interface devices described herein may also include one or more vents or leak paths 113′ (or 113, or 113″) which may communicate with the air channel through the interface body, as schematically illustrated in FIG. 1B. Leak path 113′ (or 113, or 113″) is typically openings from the air channel 103′ to the external environment (outside of the interface body 101′). A leak path (vent) may be regulated or unregulated. For example, a regulated leak path may be opened when a supply of pressurized air is provided by the source 110, and disconnecting the source may cause the vents to close (completely or partially). As mentioned above, the leak path (which may also be referred to as a PAP leak path, since it is operational during the application of PAP) may be regulated by an EPAP leak path regulator. For example, a leak path or vent may be gated by a valve (e.g., a biased, sliding gate) that is displaced to open the vents when the source 110 is connected to the connector 107′ (or 107, or 107″); detaching the source from the connector 107′ (or 107, or 107″) may allow the gate to close the leak path vent completely or partially.

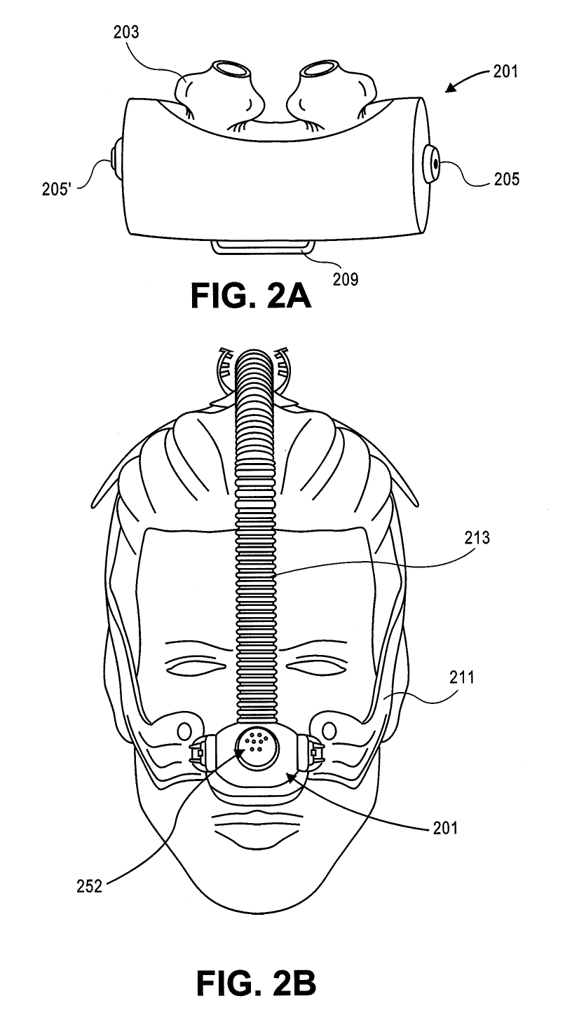

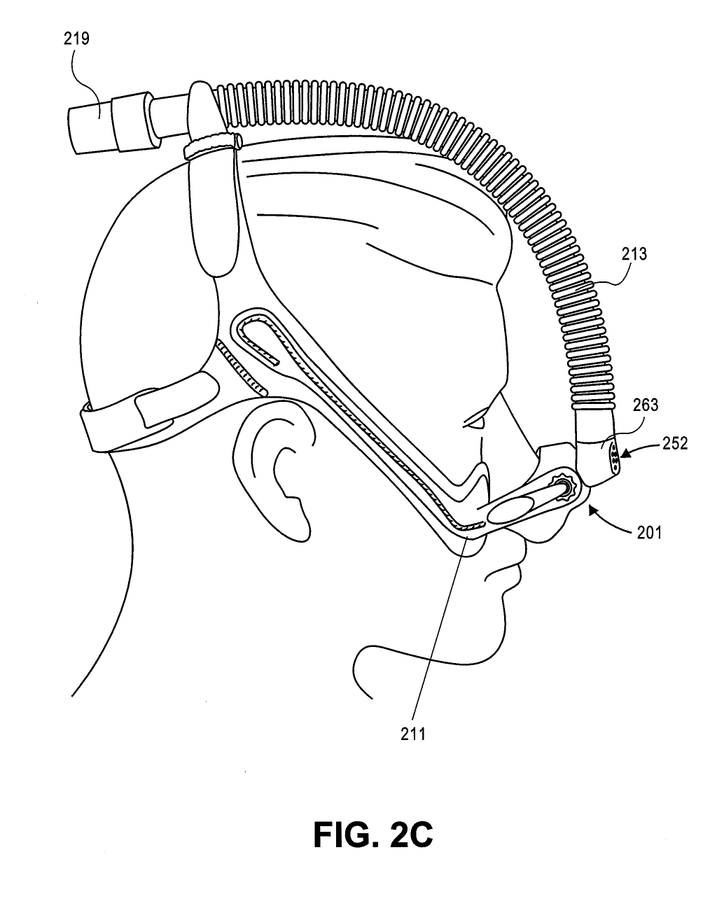

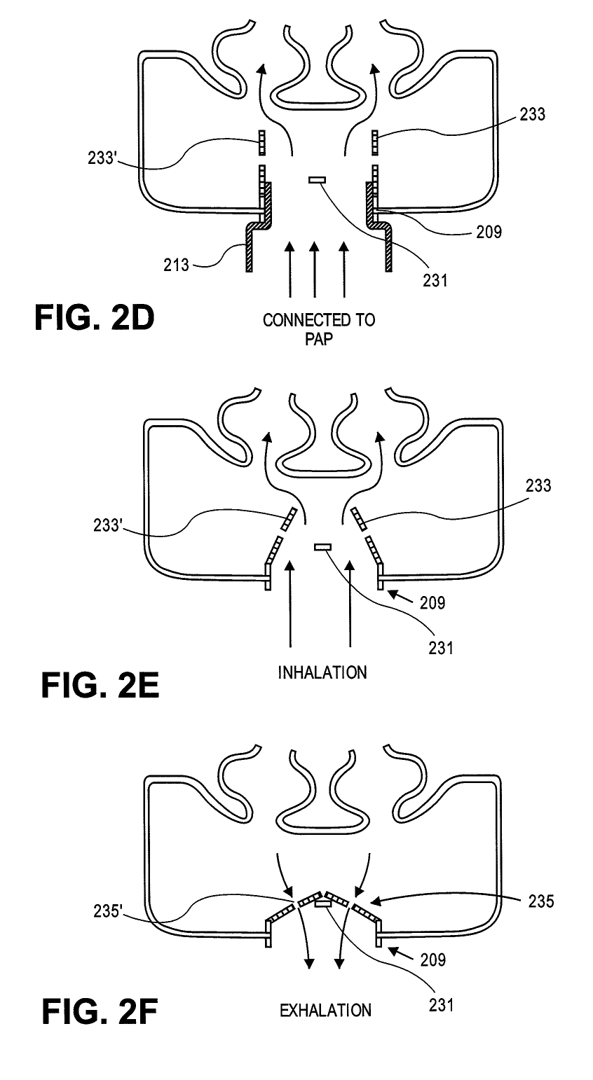

FIGS. 2A-14 illustrate variations of combined active PAP/passive EPAP interface device.

For example, FIGS. 2A-2F illustrate one variation of a combined active PAP/passive EPAP interface device configured as a nasal pillow. FIG. 2A shows a side perspective view of the interface body 201. The interface body 201 includes a user interface surface 203 that is configured as a nasal pillow for contacting the user's nose. Two openings in the nasal pillow region open into an internal air channel (not visible in FIG. 2A). A connecting region 209 configured to connect to a PAP source is located across from the user interface surface 203. As illustrated in FIGS. 2B and 2C, this variation may be secured to the user by a headset 211 that can connect to either side of the interface body 201 at connector regions 205, 205′. In FIG. 2B, the interface body 201 is shown connected to tubing 213 that may connect to a PAP source. The tubing may extend only a short distance (e.g., over the head) before connecting to a coupler that connects it to the PAP source (not shown), or it may extend from the removable connector 209 all the way to the PAP source. FIG. 2C is a side perspective view of this device.

FIGS. 2D-2F illustrate operation of the combined PAP/EPAP interface device during both the application of PAP (FIG. 2D) in the “PAP mode” and after the removal of the PAP source, by removing the tubing 213 connecting the interface to the PAP source, in the “EPAP mode.” In this example, the interface is separated from the device by pulling the tubing 213 from the interface device at the connector 209. In other variations, the PAP source may be removed while leaving at least a portion of the tubing in place (e.g., past the tubing connection 219 shown in FIG. 2C). For example, the tubing may be removed from the connector positioned behind the head 219 or further down the tubing that is connected to the interface device.

When the PAP source is connected to the combined PAP/EPAP interface, as shown in the cross-section of FIG. 2D, the EPAP airflow resistor is held inactivated. In this example, the passive EPAP airflow resistor is a flap valve having multiple flaps that are configured to open and close over the opening of the connector (as shown in greater detail in FIGS. 2E and 2F). The cross-sectional view of FIG. 2D shows two flaps forming the airflow resistor 233, 233′, although more than two (e.g., three, four, etc.) may be used. The airflow resistor includes a valve sealing surface 231. The arrows indicate the flow of air from the PAP source (not visible) through the device an out of the nasal pillow into the user's airway. The tubing connecting to the PAP source 215 is shown attached to the connector 209; securing the tubing into the connector as shown will hold the flaps of the airflow resistor open so that they cannot close when the PAP device is connected. In other variations the interface device may be configured so that the EPAP airflow resistor is secured by an EPAP activator without having to have the airflow resistor and the PAP source contact each other. For example, when the PAP device is disconnected more remotely from the interface device, the EPAP activator may be triggered to allow the EPAP airflow resistor to operate.

In FIG. 2E the PAP connection has been removed, and the airflow resistor is shown during inhalation though the device. In this variation, the airflow resistor flaps 233, 233′ are open during inhalation, allowing airflow (shown by the arrows) through the device with relatively low resistance. During exhalation, as shown in FIG. 2F, the flaps 233, 233′ close over the opening through the interface device (connector 209), against the sealing surface 231, and the air can escape only through the EPAP leak path 235, 235′. In this example, the flaps 233, 233′ of the airflow resistor each include one or more openings 235 that form the leak paths. In some variations the leak paths are formed through the wall of the interface body (see below), or in other portions of the device. Thus, during passive EPAP mode, the airflow resistor inhibits expiration more than inhalation; the resistance to exhalation is greater than the resistance to inhalation. As a result, the back pressure through the interface device during exhalation in passive EPAP mode is sufficient to induce EPAP in the user.

In this example, and in the other examples that follow, the passive EPAP airflow resistor (including the EPAP leak paths) is configured so that the resistances to inhalation and exhalation are within a predetermined therapeutic range that is sufficient to create EPAP in the user. For example, the resistance to exhalation may be between a range of about 0.001 and about 0.5 cm H₂O/(ml/sec), or between about 0.005 and about 0.25 cm H₂O/(ml/sec), or between about 0.01 and about 0.25 cm H₂O/(ml/sec) or between about 0.01 and about 0.2 cm H₂O/(ml/sec), when the resistance is measured at about 100 ml/sec.

During PAP mode, the interface device shown in FIG. 2A-2F may also include a PAP leak path, through which exhalation may occur when the PAP source is active. In the example shown in FIGS. 2A-2F, the PAP leak paths are formed by openings 252 in an intermediate (connecting) region 263 that connects the tubing 213 to the connector 209 of the interface device body 201, as shown in FIGS. 2B and 2C.

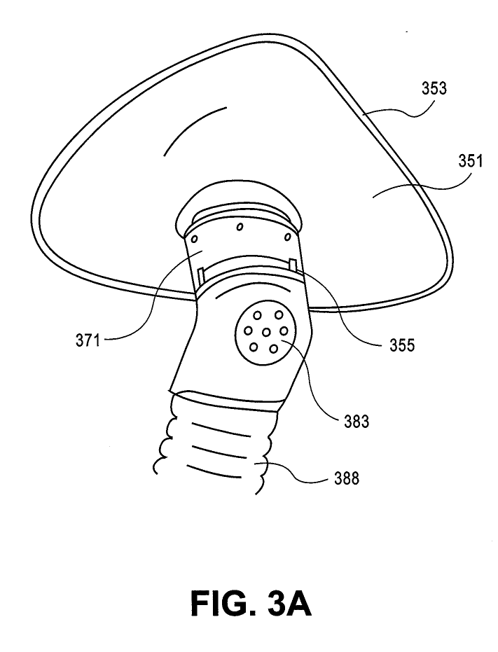

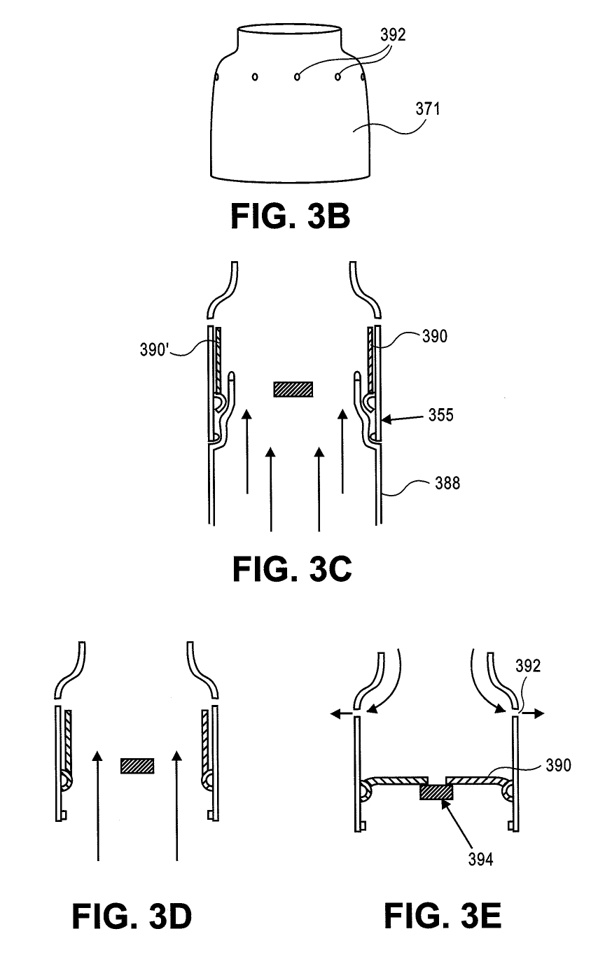

FIG. 3A-3E shows and illustrates another variation of a combined active PAP/passive EPAP device. In FIG. 3A, the interface device includes an interface body 351 and user interface surface 353 that is configured as a nasal mask. The connector and passive EPAP airflow resistor in this example are similar to the variation shown in FIGS. 2A-2F. The combined interface device shown in FIG. 3A includes a connector 355 that is connected to tubing 388, leading to the PAP source (not shown). The connector may be a quick-disconnect/quick connect (e.g., quick-release) connector, that a user can pull on to quickly release the PAP source connection.

Another feature of the embodiment shown in FIG. 3A-3E is the modular nature of the passive EPAP component. The region 371 of the combined PAP/EPAP interface device shown in FIG. 3B-3E which includes the passive EPAP airflow resistor and the EPAP leak path may be removable. Thus, this section may be an adapter device for a PAP interface, capable of converting a PAP interface into a combined active PAP/passive EPAP interface that provides passive EPAP when the PAP source is disabled. Other examples of adapters or converters are described below.

FIG. 3B shows a side perspective view of the region of the interface device 371 shown in FIG. 3A, and FIGS. 3C-3E show cross-sectional views. For example, FIG. 3C shows a cross-section through a portion of the combined interface, shown attached to a hose 388 that connects to a PAP source (not visible). The connector 355 may be a quick-release connector. When the interface device is attached to the PAP source, as shown in FIGS. 3A and 3C, the passive EPAP airflow resistor is inactivated, since the connection between the hose 388 and the device pushes against the airflow resistor, holding it open. In this example, the passive EPAP airflow resistor consists of flap valves 390, 390′ that may close during exhalation, as shown in FIG. 3E. When the connection to the PAP source has been removed, as shown in FIG. 3D and 3E, the device operates in the EPAP mode. During inhalation in the EPAP mode, the airflow resistor opens with very little resistance, as shown in FIG. 3D, allowing inhalation with very low resistance. During exhalation, the valve of the airflow resistor (the flaps in FIGS. 3A-3E) closes, and exhalation through the device is limited to the leak path, indicated as openings 392 through the body of the region 371 illustrated. The valve flaps close against a sealing surface 394 in this example. This body region may be part of the interface body, or it may be part of an adapter body that has been coupled to a PAP interface. The openings 392 visible in FIG. 3B through the body form part of the EPAP leak path, and help determine the resistance to exhalation sufficient to result in EPAP in the user (e.g., within a therapeutic range).

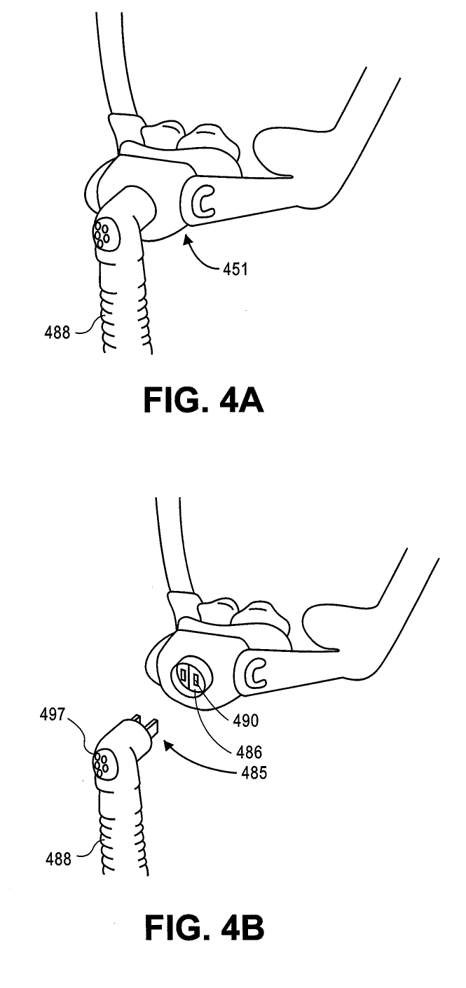

FIGS. 4A and 4B illustrate another variation of a combined active PAP/passive EPAP interface device 451 configured as a nasal pillow. In this variation, tubing 488 connecting the PAP source to the interface device includes an EPAP activator, shown as prongs 485 that disable the EPAP airflow resistor by holding it open when the tubing (and thus the PAP source) is connected to the interface device. In this example, the airflow resistor includes leak paths (openings) 490 on the valves 486 forming the airflow resistor.

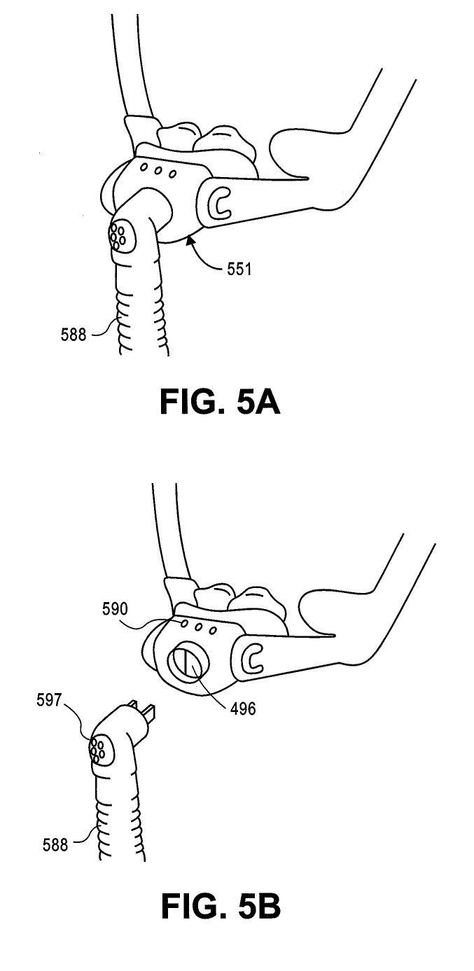

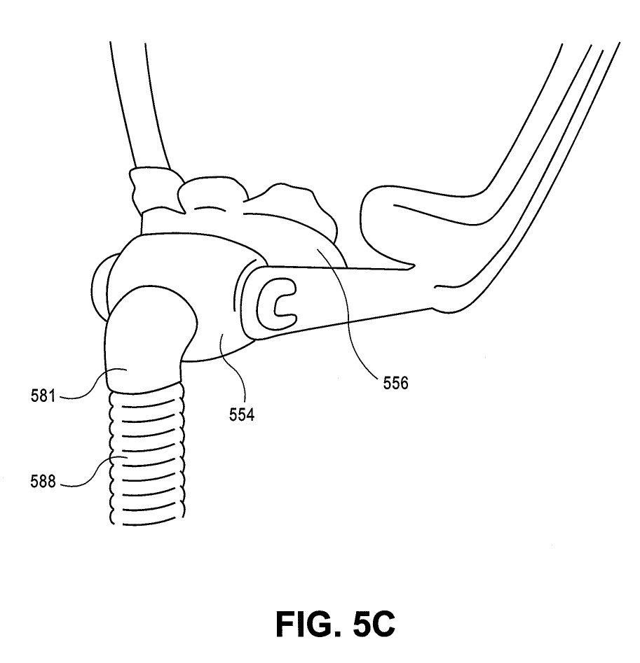

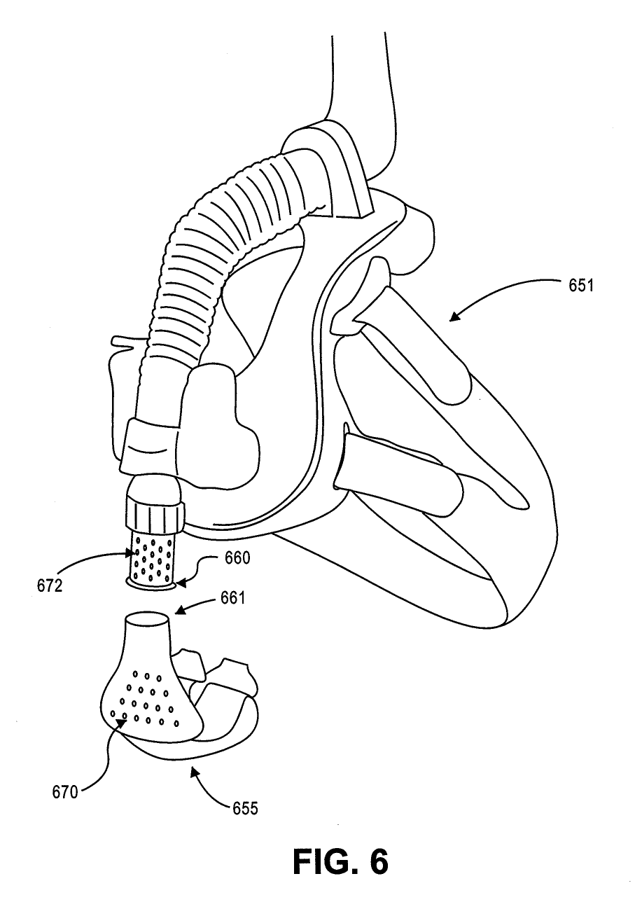

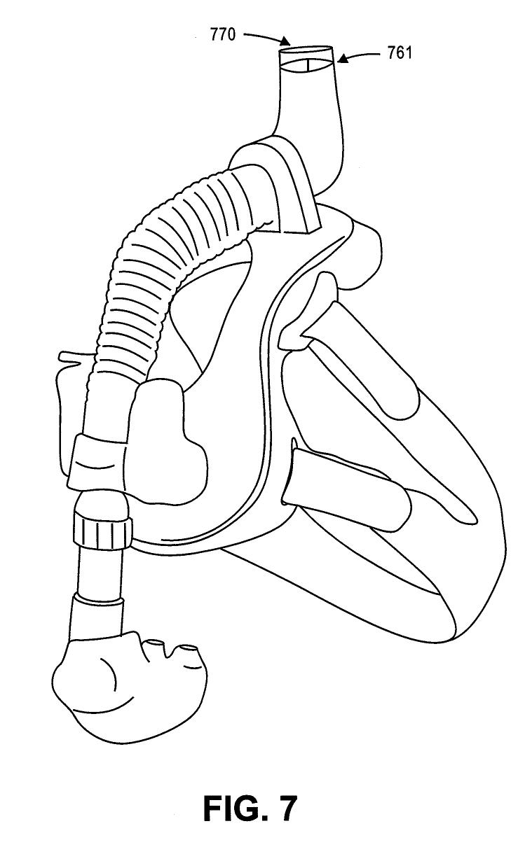

FIGS. 5A and 5B illustrate a variation of an interface device 551 similar to that shown in FIG. 4A and 4B, in which the EPAP leak paths are formed on the interface body, instead of the airflow resistor. In this example, the EPAP leak path(s) will contribute to the overall PAP leak path, which also includes the openings 497, 597 that communicate with the tubing 488, 588. In some variations the tubing itself includes these openings. Alternatively, or additionally, the openings forming the PAP leak path may be part of a separate region. For example, a connector region may be used to couple the tubing to the interface, and may include leak path openings. Thus, as illustrated in FIG. 5C, the EPAP leak path can be located on any portion of the system, including the connection to the PAP source 588, a connector region 581, the body of the interface device 554, and/or the user interface surface region 556, which is configured as a nasal pillow in this example.

FIG. 6 shows another variation of an active PAP/passive EPAP interface system, including an interface device that is configured as nasal prongs that fit inside the user's nose and may stay in place if the rest of the interface system is taken off. For example, the user may, while sleeping or trying to sleep, pull of the headpiece 651, leaving behind the PAP/EPAP interface portion 655. The connection 661 between the PAP/EPAP interface device and the rest of the system may be configured as an easy-disconnect (or quick connect/disconnect) region. In FIG. 6, this region includes a flange 660 that mates with the PAP/EPAP interface 655. When the flange 660 is connected to the connector 661 region, the EPAP airflow resistor (not visible) is inactive. Removing the connector and the rest of the headpiece 651 activates the EPAP airflow resistor (e.g., by activating an EPAP activator as described above). The PAP/EPAP interface portion may include openings through the interface body that form the EPAP leak path 670 (and part of any PAP leak path 672).

FIG. 7 illustrates a similar variation in which the device is configured to be removed from the PAP source near the tubing 770 connection to the rest of the PAP source. In this variation, the passive EPAP airflow resistor 761 is located near the release point 770 for the PAP source. Alternatively, the airflow resistor could be located closer to the user interface surface, but the EPAP activator may be located distally, near the release point for the PAP source 770. For example, the EPAP activator may include a tendon, wire, rod, or other structure that is pushed proximally, bracing open the EPAP airflow resistor, when the PAP source is connected. Alternatively, the EPAP activator may electrically or magnetically relay and/or control the passive EPAP airflow resistor when the device is connected. For example, the EPAP activator may wirelessly monitor and control/communicate from any location on the interface system the status of the PAP source (running/not running, connected/disconnected). Although we describe primarily simple mechanical EPAP activators herein, such electrical, magnetic, and other EPAP activators for controlling the interface device are also contemplated and may be used to activate the EPAP airflow resistor (and/or the EPAP leak path regulator) and toggle between the active PAP mode and the passive EPAP mode.

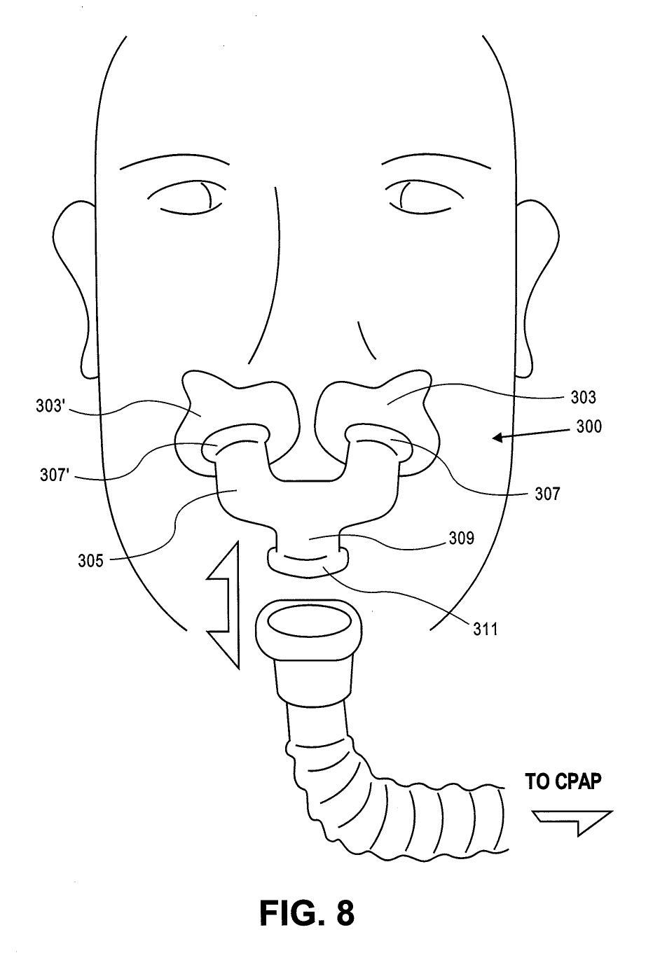

Another example of a PAP interface device is shown in FIG. 8. In this example, the combined PAP/EPAP interface device is secured on a user's nose. The interface device 300 is adhesively secured over the user's nostrils. The user interface surface(s) on the interface 303, 303′ are adhesive and can be used to secure the devices to the user's nose. In this example (not shown to scale), each nostril is adhesively secured to a separate tube which connects to a central passageway (air channel) in the interface body region of the device 305. This airway interface region may be adjustable (e.g., to adjust to the spacing between the user's nostrils, the angle of the nostrils, etc.). In some variations, both nostrils communicate with a single tube or opening into the air channel. For example, a mask covering both nostrils, the entire nose, or the nose and mouth, may be used; the mask may also be adhesively secured to the user. Other attachment means may be used in addition or alternatively. For example, a strap, tie, band, elastic tether or the like may be used to secure the interface device to the user. In some variations, this may form a seal with the user.

A connector 311 is shown at the base of the interface body region, which opens into the passageway of the airway interface 305, and may connect to the source of pressurized breathable gas (e.g., CPAP source), as indicated. In some variations an airflow resistor (not visible in FIG. 8) spans the connector 309 before it opens, so that expiration through the connector is metered. As mentioned, when the CPAP source is connected to the connector 311, it may disable the airflow resistor. Alternatively, the airflow resistor may be located some distance from the connector 311. For example, two airflow resistors, each one configured to communicate with a nostril, may be positioned near the user contacting region(s) 307, 307′. These airflow resistors may also be inactivated or disabled when the CPAP source is connected.

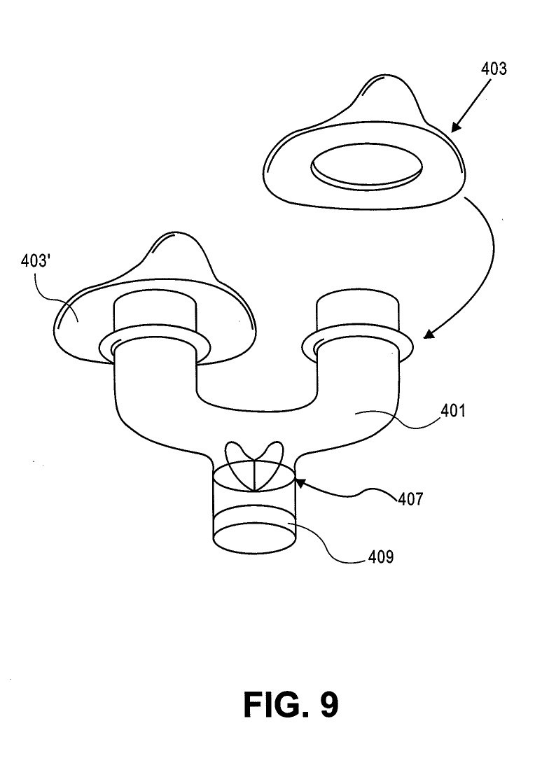

FIG. 9 shows a similar variation of a PAP interface, in which the interface body region 401 includes an adhesive user contact surface 403, 403′. The adhesive surface may be covered by a protective cover that can be removed to expose the adhesive so that the interface device can be applied to the user. In this example, the airway interface is bifurcated, and each nostril is connected to a portion of the passageway within the interface device, and a single airflow resistor 407 is positioned within the passageway (air channel). The connector 409 opens into the passageway through the airflow resistor 407. In some variations, attaching the source of pressurized breathable gas (e.g., via a tube or hose) to the connector 409 will inactivate the EPAP airflow resistor 407 by disabling the valve of the airflow resistor in the open position forming the airflow resistor.

In the example shown in FIG. 9, the airway interface portion of the device is reusable. The adhesive user contacting surfaces 403, 403′ may be single-use, disposable contact surfaces. New (e.g., replacement) adhesive user contacting surfaces 403, 403′ can be secured to the reusable airway interface portion, and the device may then be reused.

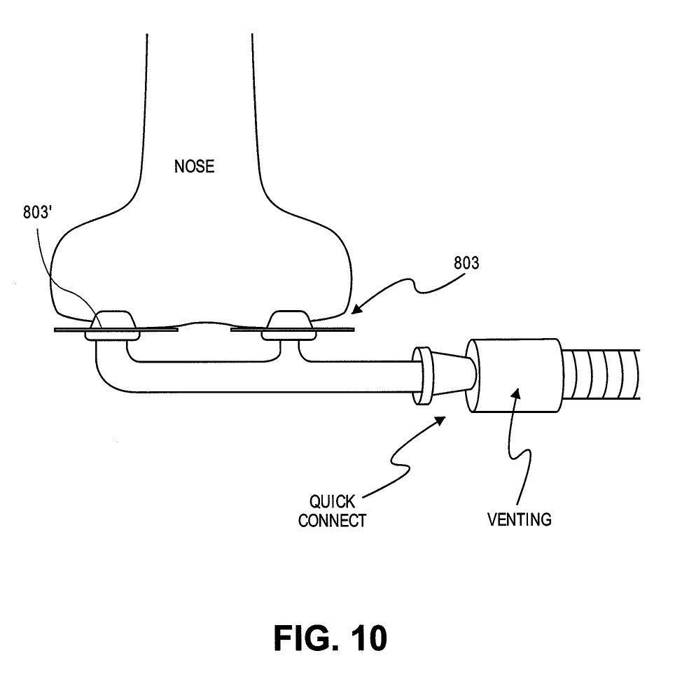

FIG. 10 schematically illustrates another variation of a combined active PAP/passive EPAP interface device, in which each user interface surface 803, 803′ includes an airflow resistor and an adhesive user contacting surface. The airflow resistor is part of the user contacting surface, so that if the device is removed, the passive EPAP airflow resistor is adhesively secured to the user's nose, and left in place, regardless of where the device is removed. As in the previous examples, the EPAP airflow resistor is inactive until the PAP source is removed or disabled. Another example of this is shown below.

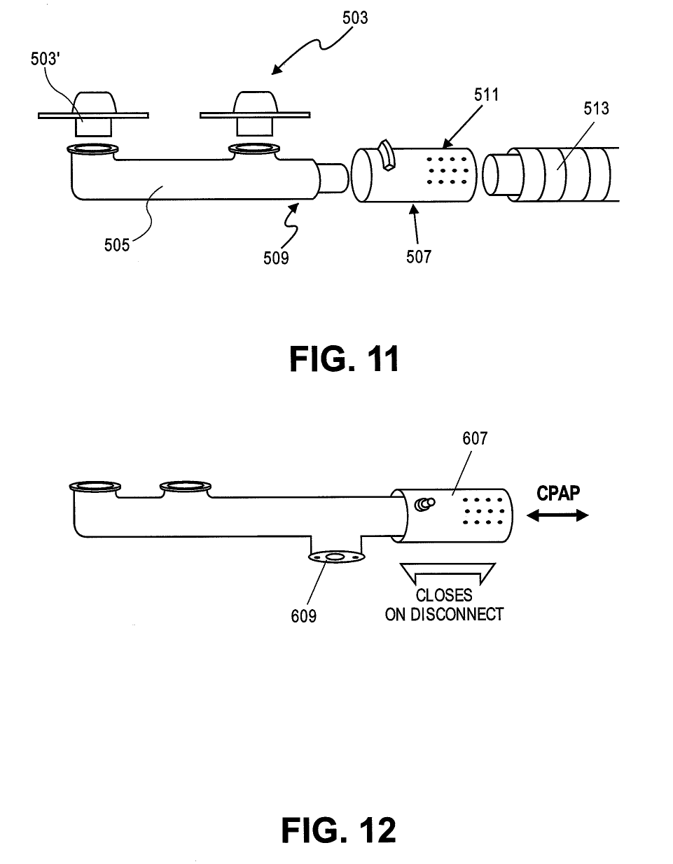

The PAP backup interface device shown in FIG. 11 is similar to that described above for FIG. 9. In this example, the various regions of the device are modular, and may connect together. For example, adhesive user interface surfaces 503, 503′ are part of a user contacting section (or sections) and may be single-use adhesive components. Each adhesive user contacting surface 503, 503′ may attach to one of a user'nostrils. The user contacting sections including the user contacting surfaces 503, 503′ can attach to an interface body region 505 including an internal air channel, as shown. A quick release/quick connect connector region 507 may be attached to the airway interface region 505. This region may include one or more vents 511 or venting regions open to the external environment, and may be configured to connect to the source of pressurized breathable gas (shown here as a tube 513).

The airflow resistor (or resistors) may be included in any of the regions described above, including the user contacting section(s), the interface body region, or the connector region. For example, an airway resistor 509 may be included as part of each user contacting section 503, 503′, or as part of the airway interface region 505. In the variation shown in FIG. 11, the airflow resistor is located at the distal end of the airway interface region 505, which connects to the connector region 507. The different regions shown disconnected in FIG. 5 may be connected permanently or removably.

In FIG. 11, the airflow resistor is configured in-line with the central air channel of the interface body region. For example, the airflow from the PAP source must pass through the airflow resistor (which may be active or inactive) in order to enter the user's airways. FIG. 12 illustrates a similar variation in which the airflow resistor 609 is connected to the air channel through the device in parallel with the opening of the connector 607.

In FIG. 12, the connector is gated (or valved), so that when the PAP source is connected, the valve is open, and when the PAP source is disconnected, the connector opening to the air channel is substantially (or completely) closed. This at least partially limits the airflow through the airway interface region of the interface device to passing through the airflow resistor 609, which increases the resistance during expiration more than inspiration. In this example, the airflow resistor is not inactivated, but can remain activated (operable) at all times. Because the airflow from the PAP source is in parallel to the airflow resistor, it does not substantially affect the resistance through the air channel from the user's airways until the connector opening is closed off.

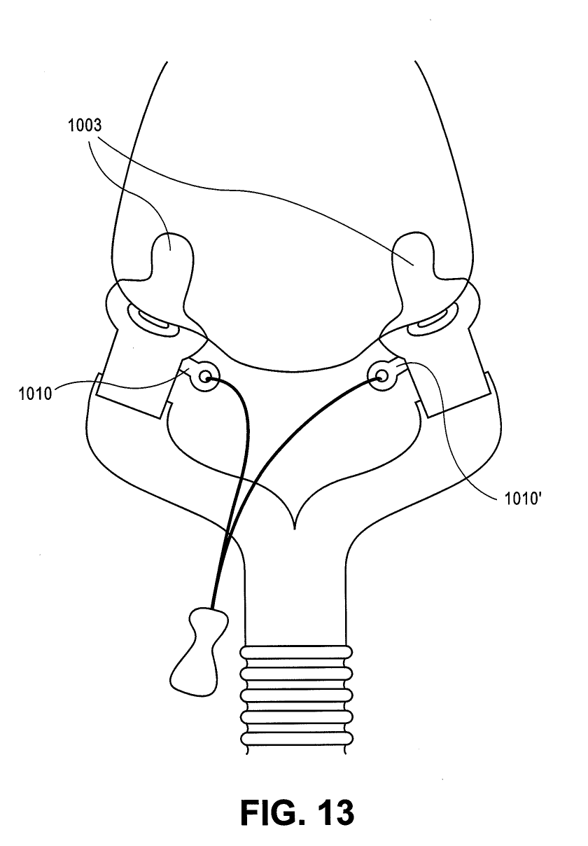

Any appropriate connector may be used to connect the interface devices described herein with the source of pressurized breathable air. In particular, quick connect/quick disconnect connectors are of particular interest. For example, FIG. 13 illustrates one variation of a PAP interface device including a quick release for the connector. In FIG. 13, the device includes a quick release “rip cord” that may be pulled by the user (e.g., when roused from sleeping, etc.) to quickly remove the CPAP source from the interface device. For example, pulling on the quick release rip cord pulls out pins 1010, 1010′ that disengage the airway interface from the user contacting sections 1003. Each user contacting section includes an airflow resistor, so that when the rip cord is pulled, the passive EPAP airflow resistor may be activated and free to apply an increased resistance to expiration in comparison to inhalation.

Other examples of quick-release or more permanent connections between the various components (including the connection to the PAP supply) are described in more detail below.

Although the variation shown above in FIG. 13 includes a user interface surface that is adhesive, any of the devices described herein may be used with any appropriate user contacting surface, including sealing, inflatable, pliable, etc. surfaces. Adapters

As mentioned, a PAP interface device may be converted into a combined active PAP/passive EPAP interface device by the addition of an EPAP airflow resistor. For example, an adapter device, system or kit may be provided. In some variations, the adapter may be configured to have an adapter body that connects to the PAP interface device, typically in communication with the air passage through the PAP interface device. The PAP interface device may include an EPAP activator to help switch between the active PAP mode and the passive EPAP mode, based on whether or not the PAP source is applying airflow.

For example, FIG. 14 is an exploded view of a PAP interface system including an adapter device 1401 for converting the nasal pillow 1403 PAP interface into a combined active PAP/passive EPAP interface device. In this example, the adapter device 1401 includes an adapter body that at least partly covers the PAP interface device (nasal pillow 1403), and includes an EPAP airflow resistor 1405; in this variation the airflow resistor is configured as a flap valve including four flaps and a valve limiting surface (not visible in FIG. 14). The valve limiting surface prevents the valve from substantially opening during exhalation. The device also includes a plug 1413 for sealing one end of the nasal pillow 1403, directing airflow through the other end. The PAP interface device (nasal pillow 1403) including PAP leak paths (holes 1421) that are at least partially closed off by the adapter 1401. The adapter also includes additional openings forming new PAP leak path(s) 1409 that are distal to the EPAP airflow resistor. The smaller leak paths on the adapter 1419 will form the EPAP leak path, so that the airflow resistor can create a therapeutic range of resistance to exhalation during passive EPAP mode. As mentioned above, the device may include a strap or frame 1411 for wearing the device on the user's head. The connection to the PAP source 1415 will disengage the EPAP airflow resistor when connected to the adapter 1401 (and therefore the PAP interface device 1403). Moreover, this connector may be configured as a preferential site for the user to disengage the connection to the PAP source. For example, the connector may be a quick-release connector.

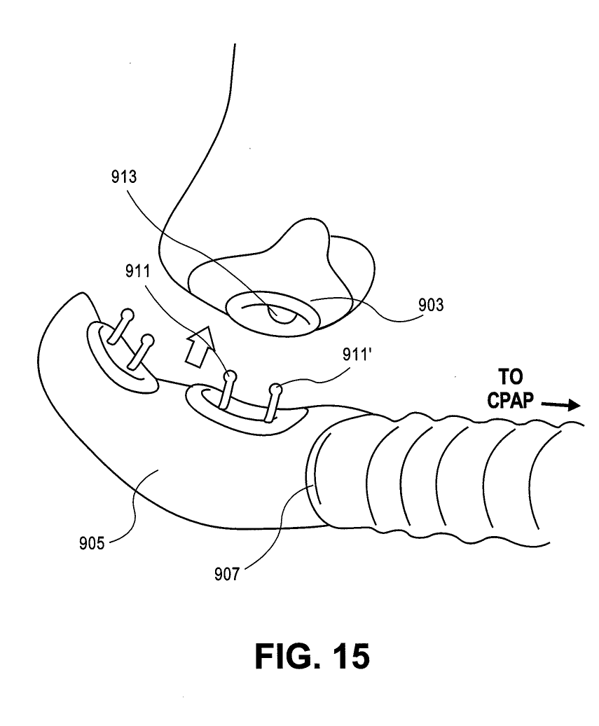

FIG. 15 illustrates another variation of a combined PAP/EPAP interface system. This variation is a two-part system, which includes an EPAP airflow resistor 913 that is adhesively secured or securable to a user's nose (as illustrated). The PAP interface device 905 then connects to the adhesively secured EPAP component 903. An adapter kit may include a nasal prong or nasal pillow that is configured to engage the adhesive EPAP component 903. In some variations, the adhesive EPAP component (which may include an EPAP airflow resistor and an EPAP leak path) is configured to accept a standard nasal prong, nasal mask, or nasal pillow. For example, the adhesive EPAP component may be configured to seal against the PAP interface. In at least one variation the EPAP component is a separate device that is worn beneath or in conjunction with the PAP interface.

In FIG. 15, the user contacting region, EPAP component 903, is adhesively attached to a user's nose, and the PAP interface 905 includes an internal air passage that is attached in communication with a connector 907 to a PAP source. The EPAP components (one may be used for each nostril, for example) 903 typically include an airflow resistor (e.g., a flap valve 913) configured to inhibit expiration more than inspiration, as just described. In some variations, the PAP interface engages with the EPAP component 903 in a quick disconnect mating; in FIG. 15, this is shown as a pair of pins on either side of a port into the air channel of the PAP interface body. Each of these pins 911, 911 ′ can removably secure the EPAP component 905 with the PAP interface 903. In addition, the pins 911, 911′ may also hold the airflow resistor (e.g., flaps) open while the PAP interface is connected, inactivating the valve(s). Thus, these pins 911, 911′ are EPAP activators. The airflow resistor may become active upon removal of the PAP interface including the pins. A sealing gasket, adhesive, or the like may also be used as part of this connection. The PAP interface body may be adjustable so that it can mate with both user contacting surface regions (one for each nostril in those variations including individual nostril attachment.

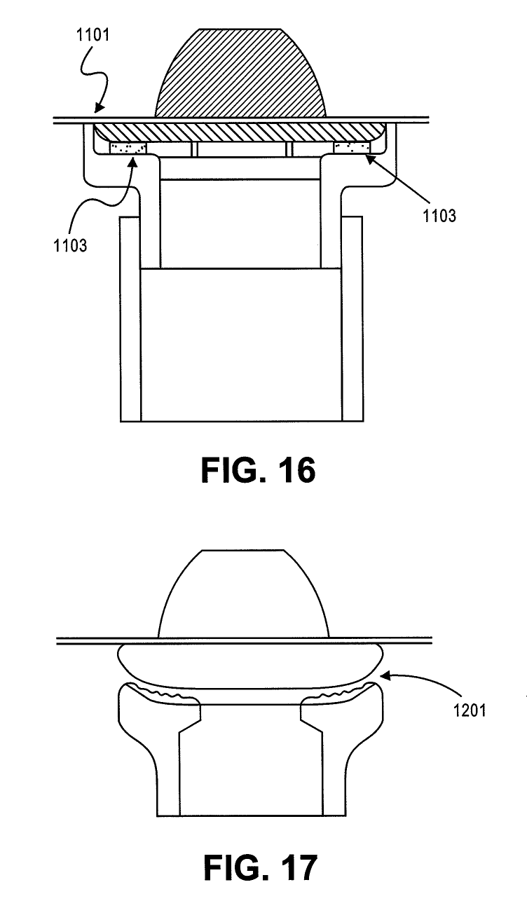

In general, the connection between the different components (and especially between the interface body and the source of CPAP) may be any appropriate connector, particularly quick release and/or quick attachment connectors. For example, FIGS. 16 and 17 illustrate two variations of quick release attachments that may be used with any of the devices herein, including the connection between the EPAP component and the PAP interface exemplified in FIG. 15, and the connection between the combined PAP/EPAP interface devices and the PAP source shown above. For example, in FIG. 16 the quick release attachment is a snap 1101. This example is shown as the connection between an EPAP component (including an airflow resistor) and the interface body of a PAP interface including a passageway, however, a similar quick-release attachment could be made between the connector region and the source of PAP. A gasket 1103 is also included to help form the seal between the two regions. Pressure applied to the snap 1101 may lock the two sections together, applying pressure on the gasket 1103 between the two. The regions may be separated by pulling them apart. For example, a user may tug on the PAP supply hose or on the airway interface to disconnect them. In some variations a button, toggle, pin, or the like may be pulled, pressed or otherwise activated to trigger release. This release mechanism may be referred to as a quick release mechanism.

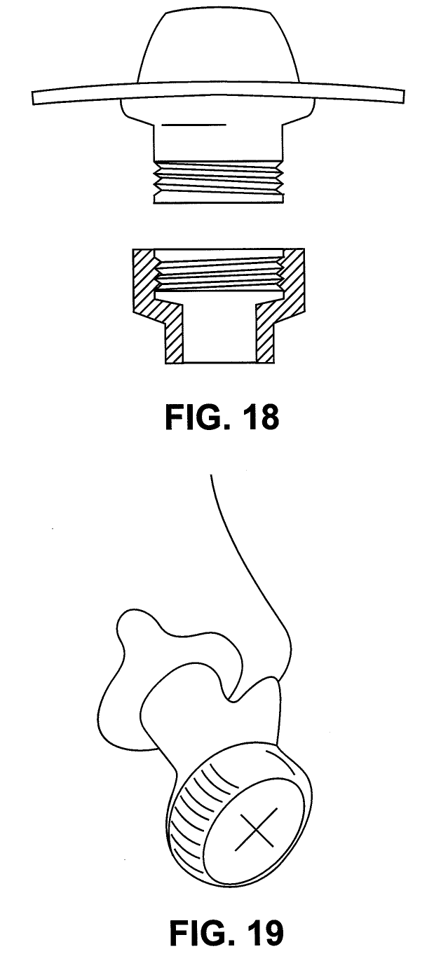

The quick release attachment shown in FIG. 17 includes an adhesive material 1201 between the two sections. For example, a first section (e.g., an EPAP component, or a connector region of a combined PAP/EPAP interface) may be friction fit to interact with a second section (e.g., the interface body of a PAP interface, or the hose from the PAP source). An adhesive material 1201 (e.g., glue or other adhesive) may line or coat one or both surfaces between the first and second sections. In some variations, a releasable material such as Velcro may be used. The adhesive or releasable connecting material 1201 is configured to have a lower release strength than the friction fit connection with the airway tubing (e.g. the hose from the PAP source) such that when the user pulls on the tube, the connection with the user interface section is broken at the adhesive bond 1201. In addition to the releasable or quick-release connections described above, in some variations one or more components may be attached more durably. For example, FIG. 18 illustrates another variation of a connector, in which the connector is a threaded screw.

In operation, the PAP backup interface devices (combined PAP/EPAP interface devices) may be worn by a user undergoing PAP therapy, such as CPAP therapy. The interface devices may be connected to the PAP source and then secured to the user, or they may first be worn by the user and then attached to the PAP device. For example, the PAP backup interface may be secured to the user so that a reasonably good seal is formed between the user and the interface device, and positive airflow can be supplied by the PAP device. In this mode of operation, active PAP airflow is provided to treat the user, and the user may sleep while wearing the device to receive the benefits of the PAP therapy. Depending on the configuration of the interface device, the airflow resistor may be activated or inactivated. If the airflow resistor (or resistors) is inactivated, for example, because the PAP source is connected to the connector on the interface device, then the airflow resistor does not contribute a substantial amount of resistance to expiration or to inspiration. This is particularly true compared to the resistance applied by the positive air flow from the PAP source.

During sleep, the user may remove the PAP source from the interface device. The PAP source may be intentionally or unintentionally removed. A user may unintentionally remove the PAP source tubing from the interface device while sleeping, partially awake or semi-conscious by pulling on the tubing supplying the PAP. The airway interface may be configured to stay attached to the user while the connector more readily releases from the interface device. For example, the connector may be a quick release or easy release connector. A user may also intentionally disconnect the PAP source. For example, the user may pull a quick release rip-cord or other release mechanism triggering the disengaging of the source of pressurized airflow from interface device, while leaving the interface device on.

In some variations, the PAP source remains connected to the interface device, but the positive air pressure is discontinued or disabled. For example, this may occur during a power failure or PAP source failure. Thus, the combined PAP/EPAP devices may be configured to apply “backup” passive EPAP even with the PAP source (e.g., hose) connected to the interface device.

Once the PAP source has been disconnected or disabled, stopping the active application of positive pressure from the CPAP source, passive EPAP may be applied by the airflow resistor. In this mode, respiration may occur through the interface device, and more specifically through the airflow resistor. As mentioned above, the airflow resistor may be configured to provide a greater resistance to expiration than to inspiration. This greater resistance to expiration may help maintain a higher pressure in the airway prior to the following inspiration (e.g., mimicking ‘pursed lip’ breathing). In some variations, the airflow resistor may be configured to create PEEP (positive end expiratory pressure). For example, the airflow resistor may have a non-zero threshold pressure for opening during expiration so that the airflow resistor is closed during expiration when the pressure across the airflow resistor is below the threshold pressure for opening, and the airflow resistor opens during expiration when the pressure across the airflow resistor exceeds the threshold pressure for opening during expiration. Any appropriate threshold pressure for opening during expiration may be used. For example, the threshold pressure for opening (which may also be referred to as the threshold for opening) of the airflow resistor may be less than about 20 cm H₂O, less than about 15 cm H₂O, less than about 10 cm H₂O, less than about 8 cm H₂O, more than about 4 cm H₂O, or between a range of pressures. For example, the threshold pressure for opening may be between about 0.5 cm H₂O and about 20 cm H₂O, or between about 0.5 cm H₂O and about 15 cm H₂O, or between about 4 cm H₂O and about 20 cm H₂O. The threshold for opening may be less than the pressure resulting from coughing, sneezing, or the like.

In some variations, the airflow resistor may further comprise a non-zero threshold pressure for closing during expiration, such that the airflow resistor closes during expiration when the pressure across the airflow resistor falls below the threshold pressure for closing. Any appropriate threshold pressure for closing during expiration may be used. For example, the threshold pressure for closing during expiration may be greater than about 1 cm H₂O, greater than about 2 cm H₂O, greater than about 3 cm H₂O, greater than about 4 cm H₂O, greater than about 10 cm H₂O, etc. In some variations, the threshold pressure for closing during expiration is between a range of values, such as between about 0.5 cm H₂O and about 20 cm H₂O, between about 0.5 cm H₂O and about 15 cm H₂O, between about 0.5 cm H₂O and about 10 cm H₂O, between about 0.5 cm H₂O and about 5 cm H₂O. The threshold pressure for closing during expiration may be approximately the same as the threshold pressure for opening during expiration, or it may be different.

In some variations the airflow resistor of the device has a threshold pressure for opening that is less than the threshold pressure for closing. In this variation, the device opens when the pressure exceeds the threshold for opening (e.g., at 4 cm H₂O), and then closes at a predetermined time after opening after which the pressure must reach a second threshold for opening (e.g., at 10 cm H₂O). This may allow a user to breathe out easily at first (possibly improving tolerance for the device) and then have a larger PEEP pressure at the end of expiration.

The passive resistance to expiration provided by the airflow resistor contrasts with the active resistance to expiration provided by the source of positive airflow (a PAP source). The PAP interface devices described herein effectively include a “backup” for maintaining the patency of the user's airways while they are wearing the interface, even in the absence of the applied positive pressure, because the airflow resistor at least partially regulates expiration through the interface device.

The interface device does not need to form a complete seal with the user's airway. For example, the leak paths or vents on the interface permit some airflow to/from the external environment (outside of the interface device). As long as the resistance to expiration can be increased during expiration by the airflow resistor, multiple (or additional) leak pathways may be present. In general, the resistance to expiration of the device when the airflow resistor is closed (the total leak pathway) is greater than the resistance to expiration when the airflow resistor is opened. In addition, the device may be configured so that the resistance to exhalation, including the contribution of any leak, is within a therapeutic range for EPAP (e.g., between about 0.001 and about 0.5 cm H₂O/(ml/sec)) when measured at a flow rate of 100 ml/sec.

The devices described herein may find use in the treatment of respiratory and non-respiratory disorders including but not limited to sleep disordered breathing, snoring, sleep apnea, obstructive sleep apnea, central sleep apnea, mixed sleep apnea, complex sleep apnea, UARS, COPD (including emphysema and chronic bronchitis), cystic fibrosis, asthma, GERD, hiatal hernia, pulmonary edema, heart failure and the like.

Although various examples have been described, many other materials and structures may be used to form a combined PAP/EPAP devices described herein. This description is not intended to be limited to the structures and materials described herein, but is also intended to encompass many other materials and structures having similar properties. Other variations of the devices described herein are, of course, possible. While the methods and devices have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A combined active PAP/passive EPAP interface device to be worn by a user to transmit positive air pressure from a PAP source to the user and to provide passive EPAP when the PAP source is disabled, the device comprising:
an interface body configured to be secured in communication with the user's airway and to connect to the PAP source;
a passive EPAP airflow resistor in communication with the interface body and configured to passively produce expiratory positive airway pressure when the PAP source is disabled.

2. The device of claim 1, further comprising a quick release mechanism configured to disconnect the PAP source from the interface body.

3. The device of claim 2, wherein the quick release mechanism comprises a pull cord.

4. The device of claim 1, further comprising a releasable connector configured to releasably connect the interface body to the PAP source for delivering pressurized breathable gas.

5. The device of claim 1, further comprising a PAP leak pathway in communication with the interface body, through which air may be exhaled when the PAP source is enabled and providing positive air pressure though the interface body.

6. The device of claim 1, further comprising an EPAP leak pathway in communication with the interface body, through which air may be exhaled when the PAP source is disabled, further wherein the EPAP leak pathway permits less airflow through the interface body during exhalation than a PAP leak pathway permits during exhalation when the PAP source is enabled and providing positive air pressure through the interface body.

7. The device of claim 1, further comprising an EPAP activator configured to inactivate the passive EPAP airflow resistor when the PAP source is enabled.

8. The device of claim 1, further comprising an EPAP activator configured to inactivate the passive EPAP airflow resistor when the PAP source is connected to the interface body.

9. The device of claim 8, wherein the EPAP activator comprises a displaceable member configured to be displaced when the PAP source is connected to the interface body.

10. The device of claim 1, further comprising a user interface surface configured to contact the user's face.

11. The device of claim 10, wherein the user interface surface is configured as a face mask, nasal pillow, nasal mask, or nasal prong.

12. The device of claim 1, wherein the interface comprises an adhesive surface configured to adhesively secure the interface body to the user.

13. The device of claim 1, further comprising a strap or frame for securing the interface body to the user's head.

14. The device of claim 1, wherein the interface is configured to communicate with the user's nose but not the user's mouth.

15. The device of claim 1, wherein the interface is configured to communicate with the user's nose and mouth.

16. The device of claim 1, wherein the passive EPAP airflow resistor comprises a flap valve.

17. The device of claim 1, wherein the passive EPAP airflow resistor includes a valve that is selected from the group consisting of: ball valve, flap valve, membrane valve, hingeless valve, balloon valve, duck-bill valve, PEEP valve, threshold valve and stopper valve.

18. The device of claim 1, wherein the passive EPAP airflow resistor provides a resistance to exhalation through the interface body when the PAP source is disabled that is between about 0.001 and about 0.5 cm H₂O/(ml/sec) when the resistance is measured at 100 ml/sec.

19. The device of claim 1, wherein the passive EPAP airflow resistor provides a resistance to exhalation through the interface body when the PAP source is disabled that is between about 0.005 and about 0.25 cm H₂O/(ml/sec) when the resistance is measured at 100 ml/sec.

20. The device of claim 1, wherein the passive EPAP airflow resistor provides a resistance to exhalation through the interface body when the PAP source is disabled that is between about 0.01 and about 0.25 cm H₂O/(ml/sec) when the resistance is measured at 100 ml/sec.

21. The device of claim 1, further comprising an airflow resistor bypass configured to transiently decrease the resistance to air exhaled through the passive EPAP airflow resistor for a delay period, after activation of the airflow resistor bypass.

22. A combined active PAP/passive EPAP interface device to be worn by a user to transmit positive air pressure from a PAP source to the user and to provide passive EPAP when the PAP source is disabled, the device comprising:
an interface body having an air channel, wherein the interface body is configured to secure the air channel in communication with a user's airway;
a connector configured to connect the air channel to a PAP source; and
a passive EPAP airflow resistor configured to inhibit exhalation through the interface device more than inhalation through the interface device when the PAP source is not providing pressurized breathable gas through the interface device, wherein the passive EPAP airflow resistor provides a resistance to exhalation through the interface device that is between about 0.001 and about 0.5 cm H₂O/(ml/sec) when the resistance is measured at 100 m/sec.

23. The device of claim 22, wherein the passive EPAP airflow resistor provides a resistance to exhalation through the interface device when the PAP source is disabled that is between about 0.005 and about 0.25 cm H₂O/(ml/sec) when the resistance is measured at 100 ml/sec.

24. The device of claim 22, wherein the passive EPAP airflow resistor provides a resistance to exhalation through the interface device when the PAP source is disabled that is between about 0.01 and about 0.25 cm H₂O/(ml/sec) when the resistance is measured at 100 ml/sec.

25. The device of claim 22, further comprising a user interface surface configured to contact the user's face, wherein the user interface surface is configured as a face mask, nasal pillow, nasal mask, or nasal prong.

26. The device of claim 22, wherein the interface device is configured to communicate with the user's nose but not the user's mouth.

27. The device of claim 22, wherein the interface device is configured to communicate with the user's nose and mouth.

28. The device of claim 22, further comprising a strap or frame for securing the interface body to the user's head.

29. The device of claim 22, wherein the connector comprises a quick release connector.

30. The device of claim 22, wherein the passive EPAP airflow resistor comprises a flap valve.

31. The device of claim 22, wherein the passive EPAP airflow resistor includes a valve that is selected from the group consisting of: ball valve, flap valve, membrane valve, hingeless valve, balloon valve, duck-bill valve, PEEP valve, threshold valve and stopper valve.

32. The device of claim 22, further comprising an EPAP leak pathway in communication with the interface body, through which air may be exhaled when the PAP source is disabled, further wherein the EPAP leak pathway permits less airflow through the interface body during exhalation than a PAP leak pathway permits during exhalation when the PAP source is enabled and providing positive air pressure through the interface body.

33. The device of claim 22, further comprising an EPAP activator configured to inactivate the passive EPAP airflow resistor when the PAP source is enabled.

34. The device of claim 22, further comprising an EPAP activator configured to inactivate the passive EPAP airflow resistor when the PAP source is connected to the interface body.

35. The device of claim 34, wherein the EPAP activator comprises a displaceable member configured to be displaced when the PAP source is connected to the interface body.

36. A combined active PAP/passive EPAP interface device to be worn by a user to transmit positive air pressure from a PAP source to the user and to provide passive EPAP when the PAP source is disabled, the device comprising:
an interface body having an air channel that is configured to be secured in communication with a user's airway;
a connector configured to connect to a PAP source in communication with the air channel;
a PAP leak pathway through which air is exhaled when the PAP source is connected in communication with the air channel;
a passive EPAP airflow resistor configured to inhibit exhalation through the air channel more than inhalation through the air channel when the PAP source is disabled; and
an EPAP leak pathway through which air is exhaled when the PAP source is disabled, wherein the PAP leak pathway allows greater airflow than the EPAP leak pathway.

37. The device of claim 36, further comprising a user interface surface configured to contact the user's face, wherein the user interface surface is configured as a face mask, nasal pillow, nasal mask, or nasal prong.

38. The device of claim 36, wherein the interface is configured to communicate with the user's nose but not the user's mouth.

39. The device of claim 36, wherein the interface is configured to communicate with the user's nose and mouth.

40. The device of claim 36, further comprising a strap or frame for securing the interface body to the user's head.

41. The device of claim 36, wherein the connector comprises a quick release connector.

42. The device of claim 36, wherein the passive EPAP airflow resistor comprises a flap valve.

43. The device of claim 36, wherein the passive EPAP airflow resistor includes a valve that is selected from the group consisting of: ball valve, flap valve, membrane valve, hingeless valve, balloon valve, duck-bill valve, PEEP valve, threshold valve and stopper valve.

44. The device of claim 36, wherein the passive EPAP airflow resistor is configured to provide a resistance to exhalation through the interface body when the PAP source is disabled that is between about 0.001 and about 0.5 cm H₂O/(ml/sec) when the resistance is measured at 100 ml/sec.

45. The device of claim 36, wherein the passive EPAP airflow resistor is configured to provide a resistance to exhalation through the interface body when the PAP source is disabled that is between about 0.005 and about 0.25 cm H₂O/(ml/sec) when the resistance is measured at 100 ml/sec.

46. The device of claim 36, wherein the passive EPAP airflow resistor is configured to provide a resistance to exhalation through the interface body when the PAP source is disabled that is between about 0.01 and about 0.25 cm H₂O/(ml/sec) when the resistance is measured at 100 ml/sec.

47. The device of claim 36, further comprising an EPAP activator configured to inactivate the passive EPAP airflow resistor when the PAP source is enabled.

48. The device of claim 36, further comprising an EPAP activator configured to inactivate the passive EPAP airflow resistor when the PAP source is connected to the interface body.

49. The device of claim 48, wherein the EPAP activator comprises a displaceable member configured to be displaced when the PAP source is connected to the interface body.

50. A combined active PAP/passive EPAP interface device to be worn by a user to transmit positive air pressure from a PAP source to the user and to provide passive EPAP when the PAP source is disabled, the device comprising:
an interface body having an air channel, wherein the interface body is configured to secure the air channel in communication with a user's airway;
a connector configured to connect the air channel to a PAP source;
a passive EPAP airflow resistor in communication with the air channel; and
an EPAP activator configured to activate the passive EPAP airflow resistor when the source of PAP is disabled and to inactivate the passive EPAP airflow resistor when the source of PAP is enabled.

51. The device of claim 50, further comprising a user interface surface configured to contact the user's face, wherein the user interface surface is configured as a face mask, nasal pillow, nasal mask, or prong.

52. The device of claim 50, wherein the interface is configured to communicate with the user's nose but not the user's mouth.

53. The device of claim 50, wherein the interface is configured to communicate with the user's nose and mouth.

54. The device of claim 50, further comprising a strap or frame for securing the interface body to the user's head.

55. The device of claim 50, wherein the connector comprises a quick release connector.

56. The device of claim 50, wherein the passive EPAP airflow resistor comprises a flap valve.

57. The device of claim 50, wherein the passive EPAP airflow resistor includes a valve that is selected from the group consisting of: ball valve, flap valve, membrane valve, hingeless valve, balloon valve, duck-bill valve, PEEP valve, threshold valve and stopper valve.

58. The device of claim 50, wherein the passive EPAP airflow resistor is configured to provide a resistance to exhalation through the interface body when the PAP source is disabled that is between about 0.001 and about 0.5 cm H₂O/(ml/sec) when the resistance is measured at 100 ml/sec.

59. The device of claim 50, wherein the passive EPAP airflow resistor is configured to provide a resistance to exhalation through the interface body when the PAP source is disabled that is between about 0.005 and about 0.25 cm H₂O/(ml/sec) when the resistance is measured at 100 ml/sec.

60. The device of claim 50, wherein the passive EPAP airflow resistor is configured to provide a resistance to exhalation through the interface body when the PAP source is disabled that is between about 0.01 and about 0.25 cm H₂O/(ml/sec) when the resistance is measured at 100 ml/sec.

61. The device of claim 50, wherein the EPAP activator is configured to inactivate the passive EPAP airflow resistor when the PAP source is connected to the interface body.

62. The device of claim 50, wherein the EPAP activator comprises a displaceable member configured to be displaced when the PAP source is connected to the interface body.

63. An adapter device for a PAP interface that connects to a PAP source, the adapter device capable of converting the PAP interface into a combined active PAP/passive EPAP interface that provides passive EPAP when the PAP source is disabled, the device comprising:
a passive EPAP airflow resistor configured to be placed in communication with an airway through the PAP interface to passively inhibit exhalation more than inhalation, wherein the passive EPAP airflow resistor is configured to provide a resistance to exhalation when the PAP source is disabled that is between about 0.001 and about 0.5 cm H₂O/(ml/sec) when the resistance is measured at 100 ml/sec.

64. An adapter device for a PAP interface that connects to a PAP source, the adapter device capable of converting the PAP interface into a combined active PAP/passive EPAP interface that provides passive EPAP when the PAP source is disabled, the device comprising:
a passive EPAP airflow resistor configured to be placed in communication with an airway to passively inhibit exhalation more than inhalation through the PAP interface and produce expiratory positive airway pressure; and
an EPAP actuator configured to activate the passive EPAP airflow resistor when the PAP source is disabled and to inactivate the passive EPAP airflow resistor when the PAP source is enabled.

65. The adapter device of claim 64, further comprising an adapter body having an air passage that is configured to be placed in communication with the airway through the PAP interface.

66. The adapter device of claim 64, wherein the EPAP actuator is configured to inactivate the passive EPAP airflow resistor when the PAP source is connected to the PAP interface.

67. The device of claim 64, wherein the EPAP activator comprises a displaceable member configured to be displaced when the PAP source is connected to the PAP interface.

68. The device of claim 64, wherein the EPAP activator comprises a sensor to determine when the flow of positive pressure from the PAP source has been discontinued.

69. The device of claim 64, further comprising an EPAP leak path regulator configured to reduce the exhalation leak pathway through the device when the PAP source is disabled.

70. The device of claim 64, wherein the passive EPAP airflow resistor is configured to provide a resistance to exhalation through the PAP interface when the PAP source is disabled that is between about 0.001 and about 0.5 cm H₂O/(ml/sec) when the resistance is measured at 100 ml/sec.

71. The device of claim 64, wherein the passive EPAP airflow resistor is configured to provide a resistance to exhalation through the PAP interface when the PAP source is disabled that is between about 0.005 and about 0.25 cm H₂O/(ml/sec) when the resistance is measured at 100 ml/sec.

72. The device of claim 64, wherein the passive EPAP airflow resistor is configured to provide a resistance to exhalation through the PAP interface when the PAP source is disabled that is between about 0.01 and about 0.25 cm H₂O/(mil/sec) when the resistance is measured at 100 ml/sec.

73. An adapter device for a PAP interface that connects to a PAP source, the adapter device capable of converting the interface into a combined active PAP/passive EPAP interface that provides passive EPAP when the PAP source is disabled, the device comprising:
a passive EPAP airflow resistor configured to be placed in communication with an airway to passively inhibit exhalation more than inhalation through the PAP interface and produce expiratory positive airway pressure when the PAP source is disabled;
an EPAP leak path regulator configured to reduce the exhalation leak pathway through the device when the PAP source is disabled; and
an EPAP actuator configured to activate the passive EPAP airflow resistor and the EPAP leak path regulator when the PAP source is disabled and to inactivate the passive EPAP airflow resistor and the EPAP leak path regulator when the PAP source is enabled.

74. The adapter device of claim 73, further comprising an adapter body having an air passage that is configured to be placed in communication with the airway through the PAP interface.

75. The adapter device of claim 73, wherein the EPAP actuator is configured to inactivate the passive EPAP airflow resistor when the PAP source is connected to the PAP interface.

76. The device of claim 73, wherein the EPAP activator comprises a displaceable member configured to be displaced when the PAP source is connected to the PAP interface.

77. The device of claim 73, wherein the EPAP activator comprises a sensor to determine when the flow of positive pressure from the PAP source has been discontinued.

78. The device of claim 73, wherein the passive EPAP airflow resistor is configured to provide a resistance to exhalation through the PAP interface when the PAP source is disabled that is between about 0.001 and about 0.5 cm H₂O/(ml/sec) when the resistance is measured at 100 ml/sec.

79. The device of claim 73, wherein the passive EPAP airflow resistor is configured to provide a resistance to exhalation through the PAP interface when the PAP source is disabled that is between about 0.005 and about 0.25 cm H₂O/(ml/sec) when the resistance is measured at 100 ml/sec.

80. The device of claim 73, wherein the passive EPAP airflow resistor is configured to provide a resistance to exhalation through the PAP interface when the PAP source is disabled that is between about 0.01 and about 0.25 cm H₂O/(ml/sec) when the resistance is measured at 100 ml/sec.

81. A method of treating a respiratory or sleeping disorder, the method comprising:
providing an active PAP therapy by placing a PAP source in communication with a user's airway through an active PAP/passive EPAP interface connected to the PAP source; and
passively creating EPAP in the user using the active PAP/passive EPAP interface when the PAP source is disabled.

82. The method of claim 81, further comprising securing the active PAP/passive EPAP interface in communication with the user's mouth and nose.

83. The method of claim 81, further comprising securing the active PAP/passive EPAP interface in communication with the user's nose.

84. The method of claim 81, wherein the step of passively creating EPAP in the user using the active PAP/passive EPAP interface when the PAP source is disabled comprises passively creating EPAP in the user using the active PAP/passive EPAP interface when the PAP source is removed from the active PAP/passive EPAP interface.

85. The method of claim 81, wherein the step of passively creating EPAP in the user comprises activating a passive EPAP airflow resistor when the PAP source is disabled.

86. The method of claim 81, wherein the step of passively creating EPAP in the user comprises providing a resistance to exhalation through the PAP interface when the PAP source is disabled that is between about 0.001 and about 0.5 cm H2O/(ml/sec) when the resistance is measured at 100 ml/sec.

87. The method of claim 81, wherein the step of passively creating EPAP in the user comprises providing a resistance to exhalation through the PAP interface when the PAP source is disabled that is between about 0.005 and about 0.25 cm H2O/(ml/sec) when the resistance is measured at 100 ml/sec.

88. The method of claim 81, wherein the step of passively creating EPAP in the user comprises providing a resistance to exhalation through the PAP interface when the PAP source is disabled that is between about 0.01 and about 0.25 cm H2O/(ml/sec) when the resistance is measured at 100 ml/sec.

89. A method of treating a respiratory or sleeping disorder, the method comprising:
placing an active PAP/passive EPAP interface device in communication with a user's airway;
applying a positive air pressure through the interface device by placing the interface device in communication with a PAP source; and
passively creating EPAP in the user by inhibiting exhalation more than inhalation through the interface device when the positive air pressure from the PAP source is disabled.

90. The method of claim 89, wherein the step of placing an active PAP/passive EPAP interface device in communication with a user's airway comprises securing the active PAP/passive EPAP interface in communication with the user's mouth and nose.

91. The method of claim 89, wherein the step of placing an active PAP/passive EPAP interface device in communication with a user's airway comprises securing the active PAP/passive EPAP interface in communication with the user's nose.

92. The method of claim 89, wherein the step of passively creating EPAP in the user by inhibiting exhalation more than inhalation through the interface device when the positive air pressure from the PAP source is disabled comprises passively creating EPAP in the user using the active PAP/passive EPAP interface when the PAP source is removed from the active PAP/passive EPAP interface.

93. The method of claim 89, wherein the step of passively creating EPAP in the user comprises activating a passive EPAP airflow resistor when the PAP source is disabled.

94. The method of claim 89, wherein the step of passively creating EPAP in the user comprises providing a resistance to exhalation through the PAP interface when the PAP source is disabled that is between about 0.001 and about 0.5 cm H2O/(ml/sec) when the resistance is measured at 100 ml/sec.

95. The method of claim 89, wherein the step of passively creating EPAP in the user comprises providing a resistance to exhalation through the PAP interface when the PAP source is disabled that is between about 0.005 and about 0.25 cm H2O/(ml/sec) when the resistance is measured at 100 ml/sec.

96. The method of claim 89, wherein the step of passively creating EPAP in the user comprises providing a resistance to exhalation through the PAP interface when the PAP source is disabled that is between about 0.01 and about 0.25 cm H2O/(ml/sec) when the resistance is measured at 100 ml/sec.

97. A method of converting a PAP interface device into a combined active PAP/passive EPAP interface device, the method comprising:
providing a PAP interface device configured to connect to a PAP source; and
attaching a passive EPAP airflow resistor in communication with an airway so that the passive EPAP airflow resistor passively inhibits exhalation more than inhalation to create EPAP in a user when a PAP source is not applying positive air pressure through the interface to the user.

98. The method of claim 97, further comprising attaching an EPAP actuator to the PAP interface device, wherein the EPAP actuator is configured to activate the passive EPAP airflow resistor when the PAP source is disabled and to inactivate the passive EPAP airflow resistor when the PAP source is enabled.

99. The method of claim 97, further comprising attaching a leak path regulator to the PAP interface device, wherein the leak path regulator is configured to reduce the exhalation leak pathway through the device when the PAP source is disabled.

100. The method of claim 97, wherein the step of attaching a passive EPAP airflow resistor in communication with an airway comprises securing a passive EPAP airflow resistor that its configured to provide a resistance to exhalation when the PAP source is disabled that is between about 0.001 and about 0.5 cm H₂O/(ml/sec) when the resistance is measured at 100 ml/sec.

101. The method of claim 97, wherein the step of attaching a passive EPAP airflow resistor in communication with an airway comprises securing a passive EPAP airflow resistor that its configured to provide a resistance to exhalation when the PAP source is disabled that is between about 0.005 and about 0.25 cm H₂O/(ml/sec) when the resistance is measured at 100 ml/sec.

102. The method of claim 97, wherein the step of attaching a passive EPAP airflow resistor in communication with an airway comprises securing a passive EPAP airflow resistor that its configured to provide a resistance to exhalation when the PAP source is disabled that is between about 0.01 and about 0.25 cm H₂O/(ml/sec) when the resistance is measured at 100 ml/sec.

103. A method of converting a PAP interface device into a combined active PAP/passive EPAP interface device, the method comprising:
attaching a passive EPAP airflow resistor in communication with an airway so that the passive EPAP airflow resistor passively inhibits exhalation more than inhalation when a PAP source is not applying positive air pressure through the PAP interface device; and
attaching an EPAP actuator to the PAP interface device, wherein the EPAP actuator is configured to activate the passive EPAP airflow resistor when the PAP source is disabled and to inactivate the passive EPAP airflow resistor when the PAP source is enabled.

104. The method of claim 103, further comprising attaching a leak path regulator to the PAP interface device, wherein the leak path regulator is configured to reduce the exhalation leak pathway when the PAP source is disabled.

105. The method of claim 103, wherein the step of attaching the passive EPAP airflow resistor in communication with an airway comprises securing a passive EPAP airflow resistor that its configured to provide a resistance to exhalation when the PAP source is disabled that is between about 0.001 and about 0.5 cm H₂O/(ml/sec) when the resistance is measured at 100 ml/sec.

106. The method of claim 103, wherein the step of attaching the passive EPAP airflow resistor in communication with an airway comprises securing a passive EPAP airflow resistor that its configured to provide a resistance to exhalation when the PAP source is disabled that is between about 0.005 and about 0.25 cm H₂O/(ml/sec) when the resistance is measured at 100 ml/sec.

107. The method of claim 103, wherein the step of attaching the passive EPAP airflow resistor in communication with an airway comprises securing a passive EPAP airflow resistor that its configured to provide a resistance to exhalation when the PAP source is disabled that is between about 0.01 and about 0.25 cm H₂O/(ml/sec) when the resistance is measured at 100 ml/sec.
